SEQUENCE LISTING (77 sequences)

SEQ ID NO: 1 - protein, 19 aa, Homo sapiens
KQFIENGSEFAQKLLKKFS

SEQ ID NO: 2 - protein, 25 aa, Homo sapiens
KQFIENGSEFAQKLLKKFSLLKPWA

SEQ ID NO: 3 - protein, 138 aa, Homo sapiens
MKFTTLLFLAAVAGALVYAEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 4 - protein, 119 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 5 - protein, 114 aa, Homo sapiens
DSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 6 - protein, 114 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSL

SEQ ID NO: 7 - protein, 109 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLL

SEQ ID NO: 8 - protein, 104 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEF

SEQ ID NO: 9 - protein, 14 aa, Homo sapiens
NGSEFAQKLLKKFS

SEQ ID NO: 10 - protein, 19 aa, Homo sapiens
  VARIANT 1, 5: Xaa = any amino acid modified with a cross-linking moiety
XQFIXNGSEFAQKLLKKFS

SEQ ID NO: 11 - protein, 19 aa, Homo sapiens
  VARIANT 1, 8: Xaa = any amino acid modified with a cross-linking moiety
XQFIENGXEFAQKLLKKFS

SEQ ID NO: 12 - protein, 19 aa, Homo sapiens
  VARIANT 2, 6: Xaa = any amino acid modified with a cross-linking moiety
KXFIEXGSEFAQKLLKKFS

SEQ ID NO: 13 - protein, 19 aa, Homo sapiens
  VARIANT 2, 9: Xaa = any amino acid modified with a cross-linking moiety
KXFIENGSXFAQKLLKKFS

SEQ ID NO: 14 - protein, 19 aa, Homo sapiens
  VARIANT 3, 7: Xaa = any amino acid modified with a cross-linking moiety
KQXIENXSEFAQKLLKKFS

SEQ ID NO: 15 - protein, 19 aa, Homo sapiens
  VARIANT 3, 10: Xaa = any amino acid modified with a cross-linking moiety
KQXIENGSEXAQKLLKKFS

SEQ ID NO: 16 - protein, 19 aa, Homo sapiens
  VARIANT 4, 8: Xaa = any amino acid modified with a cross-linking moiety
KQFXENGXEFAQKLLKKFS

SEQ ID NO: 17 - protein, 19 aa, Homo sapiens
  VARIANT 4, 11: Xaa = any amino acid modified with a cross-linking moiety
KQFXENGSEFXQKLLKKFS

SEQ ID NO: 18 - protein, 19 aa, Homo sapiens
  VARIANT 5, 9: Xaa = any amino acid modified with a cross-linking moiety
KQFIXNGSXFAQKLLKKFS

SEQ ID NO: 19 - protein, 19 aa, Homo sapiens
  VARIANT 5, 12: Xaa = any amino acid modified with a cross-linking moiety
KQFIXNGSEFAXKLLKKFS

SEQ ID NO: 20 - protein, 19 aa, Homo sapiens
  VARIANT 6, 10: Xaa = any amino acid modified with a cross-linking moiety
KQFIEXGSEXAQKLLKKFS

SEQ ID NO: 21 - protein, 19 aa, Homo sapiens
  VARIANT 6, 13: Xaa = any amino acid modified with a cross-linking moiety
KQFIEXGSEFAQXLLKKFS

SEQ ID NO: 22 - protein, 19 aa, Homo sapiens
  VARIANT 7, 11: Xaa = any amino acid modified with a cross-linking moiety
KQFIENXSEFXQKLLKKFS

SEQ ID NO: 23 - protein, 19 aa, Homo sapiens
  VARIANT 7, 14: Xaa = any amino acid modified with a cross-linking moiety
KQFIENXSEFAQKXLKKFS

SEQ ID NO: 24 - protein, 19 aa, Homo sapiens
  VARIANT 8, 12: Xaa = any amino acid modified with a cross-linking moiety
KQFIENGXEFAXKLLKKFS

SEQ ID NO: 25 - protein, 19 aa, Homo sapiens
  VARIANT 8, 15: Xaa = any amino acid modified with a cross-linking moiety
KQFIENGXEFAQKLXKKFS

SEQ ID NO: 26 - protein, 19 aa, Homo sapiens
  VARIANT 9, 13: Xaa = any amino acid modified with a cross-linking moiety
KQFIENGSXFAQXLLKKFS

SEQ ID NO: 27 - protein, 19 aa, Homo sapiens
  VARIANT 9, 16: Xaa = any amino acid modified with a cross-linking moiety
KQFIENGSXFAQKLLXKFS

SEQ ID NO: 28 - protein, 19 aa, Homo sapiens
  VARIANT 10, 14: Xaa = any amino acid modified with a cross-linking moiety
KQFIENGSEXAQKXLKKFS

SEQ ID NO: 29 - protein, 19 aa, Homo sapiens
  VARIANT 10, 17: Xaa = any amino acid modified with a cross-linking moiety
KQFIENGSEXAQKLLKXFS

SEQ ID NO: 30 - protein, 19 aa, Homo sapiens
  VARIANT 11, 15: Xaa = any amino acid modified with a cross-linking moiety
KQFIENGSEFXQKLXKKFS

SEQ ID NO: 31 - protein, 19 aa, Homo sapiens
  VARIANT 11, 18: Xaa = any amino acid modified with a cross-linking moiety
KQFIENGSEFXQKLLKKXS

SEQ ID NO: 32 - protein, 19 aa, Homo sapiens
  VARIANT 12, 16: Xaa = any amino acid modified with a cross-linking moiety
KQFIENGSEFAXKLLXKFS

SEQ ID NO: 33 - protein, 19 aa, Homo sapiens
  VARIANT 12, 19: Xaa = any amino acid modified with a cross-linking moiety
KQFIENGSEFAXKLLKKFX

SEQ ID NO: 34 - protein, 19 aa, Homo sapiens
  VARIANT 13, 17: Xaa = any amino acid modified with a cross-linking moiety
KQFIENGSEFAQXLLKXFS

SEQ ID NO: 35 - protein, 19 aa, Homo sapiens
  VARIANT 14, 18: Xaa = any amino acid modified with a cross-linking moiety
KQFIENGSEFAQKXLKKXS

SEQ ID NO: 36 - protein, 19 aa, Homo sapiens
  VARIANT 15, 19: Xaa = any amino acid modified with a cross-linking moiety
KQFIENGSEFAQKLXKKFX

SEQ ID NO: 37 - protein, 19 aa, Homo sapiens
  VARIANT 1, 4: Xaa = any amino acid modified with a cross-linking moiety
XQFXENGSEFAQKLLKKFS

SEQ ID NO: 38 - protein, 19 aa, Homo sapiens
  VARIANT 2, 5: Xaa = any amino acid modified with a cross-linking moiety
KXFIXNGSEFAQKLLKKFS

SEQ ID NO: 39 - protein, 19 aa, Homo sapiens
  VARIANT 3, 6: Xaa = any amino acid modified with a cross-linking moiety
KQXIEXGSEFAQKLLKKFS

SEQ ID NO: 40 - protein, 19 aa, Homo sapiens
  VARIANT 4, 7: Xaa = any amino acid modified with a cross-linking moiety
KQFXENXSEFAQKLLKKFS

SEQ ID NO: 41 - protein, 19 aa, Homo sapiens
  VARIANT 5, 8: Xaa = any amino acid modified with a cross-linking moiety
KQFIXNGXEFAQKLLKKFS

SEQ ID NO: 42 - protein, 19 aa, Homo sapiens
  VARIANT 6, 9: Xaa = any amino acid modified with a cross-linking moiety
KQFIEXGSXFAQKLLKKFS

SEQ ID NO: 43 - protein, 19 aa, Homo sapiens
  VARIANT 7, 10: Xaa = any amino acid modified with a cross-linking moiety
KQFIENXSEXAQKLLKKFS

SEQ ID NO: 44 - protein, 19 aa, Homo sapiens
  VARIANT 8, 11: Xaa = any amino acid modified with a cross-linking moiety
KQFIENGXEFXQKLLKKFS

SEQ ID NO: 45 - protein, 19 aa, Homo sapiens
  VARIANT 9, 12: Xaa = any amino acid modified with a cross-linking moiety
KQFIENGSXFAXKLLKKFS

SEQ ID NO: 46 - protein, 19 aa, Homo sapiens
  VARIANT 10, 13: Xaa = any amino acid modified with a cross-linking moiety
KQFIENGSEXAQXLLKKFS

SEQ ID NO: 47 - protein, 19 aa, Homo sapiens
  VARIANT 11, 14: Xaa = any amino acid modified with a cross-linking moiety
KQFIENGSEFXQKXLKKFS

SEQ ID NO: 48 - protein, 19 aa, Homo sapiens
  VARIANT 12, 15: Xaa = any amino acid modified with a cross-linking moiety
KQFIENGSEFAXKLXKKFS

SEQ ID NO: 49 - protein, 19 aa, Homo sapiens
  VARIANT 13, 16: Xaa = any amino acid modified with a cross-linking moiety
KQFIENGSEFAQXLLXKFS

SEQ ID NO: 50 - protein, 19 aa, Homo sapiens
  VARIANT 14, 17: Xaa = any amino acid modified with a cross-linking moiety
KQFIENGSEFAQKXLKXFS

SEQ ID NO: 51 - protein, 19 aa, Homo sapiens
  VARIANT 15, 18: Xaa = any amino acid modified with a cross-linking moiety
KQFIENGSEFAQKLXKKXS

SEQ ID NO: 52 - protein, 19 aa, Homo sapiens
  VARIANT 16, 19: Xaa = any amino acid modified with a cross-linking moiety
KQFIENGSEFAQKLLXKFX

SEQ ID NO: 53 - protein, 19 aa, Homo sapiens
KQFIENGSEFAQKLAKKFS

SEQ ID NO: 54 - protein, 19 aa, Homo sapiens
KQFIENASEFAQKLLKKFS

SEQ ID NO: 55 - protein, 19 aa, Homo sapiens
KQFIENASEFAQKALKKFS

SEQ ID NO: 56 - protein, 19 aa, Homo sapiens
KQFIENGSEFAQKLLKKAS

SEQ ID NO: 57 - protein, 19 aa, Homo sapiens
KQFAENGSEFAQKLLKKFS

SEQ ID NO: 58 - protein, 19 aa, Homo sapiens
KQAIENASEFAQKLLKKFS

SEQ ID NO: 59 - protein, 19 aa, Homo sapiens
KQAIENGSEAAQKLLKKFS

SEQ ID NO: 60 - protein, 19 aa, Homo sapiens
KQFIENGSEFAQKLLKKFS

SEQ ID NO: 61 - protein, 19 aa, Homo sapiens
KQFAENGAEFAQKLLKKFS

SEQ ID NO: 62 - protein, 19 aa, Homo sapiens
KQFIENGSEFAQKALKKAS

SEQ ID NO: 63 - protein, 17 aa, Homo sapiens
KQFIENGSEFQKLKKFS

SEQ ID NO: 64 - protein, 19 aa, Homo sapiens
KQFIENGSEFAQKALKKAS

SEQ ID NO: 65 - protein, 19 aa, Homo sapiens
KQAIENASEFAQKLLKKFS

SEQ ID NO: 66 - protein, 19 aa, Homo sapiens
KQFAENGSEFAQKLLKKFS

SEQ ID NO: 67 - protein, 18 aa, Homo sapiens
KQFIENSEFAQKALKKFS

SEQ ID NO: 68 - protein, 19 aa, Homo sapiens
KQFIENASEFAQKLLKKFS

SEQ ID NO: 69 - protein, 19 aa, Homo sapiens
KQFIENGSEFAQKCLKCFS

SEQ ID NO: 70 - protein, 19 aa, Homo sapiens
KQFIENCSECAQKLLKKFS

SEQ ID NO: 71 - protein, 19 aa, Homo sapiens
KQCIECGSEFAQKLLKKFS

SEQ ID NO: 72 - protein, 19 aa, Homo sapiens
KQFCENCSEFAQKLLKKFS

SEQ ID NO: 73 - protein, 19 aa, Homo sapiens
KQFCENCSEFAQKLLKKFS

SEQ ID NO: 74 - protein, 19 aa, Homo sapiens
KQCIECGSEFAQKLLKKFS

SEQ ID NO: 75 - protein, 19 aa, Homo sapiens
KQFIENCSECAQKLLKKFS

SEQ ID NO: 76 - protein, 19 aa, Homo sapiens
KQFIENGSEFAQKLLKKFS

SEQ ID NO: 77 - protein, 19 aa, Homo sapiens
KQFIENGSEFAQKCLKCFS

(19) United States
(12) Patent Application Publication
(10) Pub. No.: US 2024/0294573 A1
(43) Pub. Date: Sep. 5, 2024

(54) CONSTRAINED PEPTIDES

(71) Applicants: TearSolutions, Inc., Charlottesville, VA (US); The University of Virginia Patent Foundation, Charlottesville, VA (US)

(72) Inventors: Thomas R. Gadek, Park City, UT (US); Paul A. Laskar, Napa, CA (US); Gordon W. Laurie, Charlottesville, VA (US)

(21) Appl. No.: 17/620,550

(22) PCT Filed: Jun. 17, 2020
(86) PCT No.: PCT/US2020/038205

§ 371 Date: Oct. 28, 2022

Related U.S. Application Data
(60) Provisional application No. 62/863,651, filed on Jun. 19, 2019.
(60) Provisional application No. 62/863,666, filed on Jun. 19, 2019.

(51) Int. Cl. C07K 14/00; A61K 9/00; A61K 9/08; A61K 38/00; A61K 47/10; A61K 47/12; C07K 7/08

(52) U.S. Cl. CPC C07K 14/001; A61K 9/0048; A61K 9/08; A61K 47/10; A61K 47/12; C07K 7/08; A61K 38/00

(57) ABSTRACT

This application generally relates to constrained salts of peptides, constrained forms of peptides, and compositions, kits, methods of using, or uses of the same.

### CROSS REFERENCE TO RELATED APPLICATION

This application is a U.S. National Phase under 35 U.S.C. § 371 of International Application No. PCT/US2020/038205, filed Jun. 17, 2020, which claims the benefit of priority under § 119(e) of U.S. Ser. No. 62/863,651 filed Jun. 19, 2019 and U.S. Ser. No. 62/863,666, filed Jun. 19, 2019, the entire contents of each are herein incorporated by reference.

### STATEMENT REGARDING FEDERALLY SPONSORED R&D

This invention was made with government support under EY024327 awarded by the National Institute of Health. The government has certain rights in the invention.

### REFERENCE TO SEQUENCE LISTING

The present application is being filed along with a Sequence Listing in electronic format. The Sequence Listing is provided as a file entitled TEAR_013NP_SEQUENCE_LISTING.TXT, was created on Jun. 28, 2022 and is 27,672 bytes in size. The information in the electronic format of the Sequence Listing is incorporated herein by reference in its entirety.

### BACKGROUND

### Field

The present application relates to the fields of chemistry, biochemistry and medicine. More particularly, several embodiments of the present application relate to constrained peptides, compositions, methods of using, and kits comprising such compositions. Specifically, several embodiments of the present application describe compositions comprising an aqueous solution comprising one or more constrained peptides.

Several embodiments of the present application also relate to constrained salts of peptides, compositions, methods of using, and kits comprising such compositions. Several embodiments of the present application also describe compositions comprising an aqueous solution comprising one or more constrained salts of peptides.

### Description of the Related Art

Polypeptides are increasingly being recognized as potential therapeutic agents. Consequently, there is an increased interest in exploring polypeptides in pharmaceutical research and development. However, polypeptides are notoriously difficult to formulate and additives used to preserve or stabilize such formulations result in, for example, undesired side effects. Polypeptide therapeutics are also particularly susceptible to degradation, both during storage and after administration (e.g., proteolysis). Moreover, the efficacy of polypeptide therapeutics is also often based on the peptide forming a secondary or tertiary structure. But short peptides usually do not retain their native conformation and binding capability, particularly when excised as a fragment of a larger protein, as they lack the structural reinforcement provided by the remainder of the protein. Peptides are also susceptible to proteolytic degradation and typically cannot insert into a lipid layer or cross the cell membrane. Indeed, such shorter peptides in particular tend to form unstructured random coils in solution, and thus have decreased efficacy and increased susceptibility to degradation.

### SUMMARY

There is an unmet need for peptide compositions that provide therapeutic amounts of peptides in their bioactive conformations, are stable at room temperature, and contain only trace amounts of stabilizers and/or preservatives, or none at all. To address such needs and others, several embodiments of the present application provide a constrained salt of a polypeptide, and pharmaceutically acceptable salts thereof. Constrained salts of a peptide, as described herein, utilize a self-associating anionic or cationic salt of an amphipathic peptide to provide a lipophilic micelle, lipid bilayer or other lipid surface to the lipophilic face of the amphipathic peptide capable of stabilizing the alpha-helical secondary structure of the peptide, thereby conserving, improving and/or restoring the biological activity of the peptide, for example, binding affinity towards the peptide's target or targets. In addition, several embodiments of the present application provide constrained peptides, and pharmaceutically acceptable salts thereof. Constraining the peptide, as described herein, comprises reinforcing the alpha-helical secondary structure of the peptide, thereby conserving, improving and/or restoring biological activity, for example, binding affinity towards the peptide's target or targets.

In some embodiments, the peptide (or combination of peptides) is constrained in the compositions of the present application so as to allow for long-term storage while maintaining efficacy and/or delivery over a prolonged period of time. As such, in some embodiments the constrained salt of a peptide, and compositions comprising a constrained salt of a peptide, are stable at non-refrigerated temperatures without the need for reconstitution, and are functional over a range of temperatures, including temperatures ranging from 0-40° C. In some embodiments the constrained salt of a peptide, and compositions comprising a constrained salt of a peptide, also provide increased biological and/or chemical stability, and/or efficacy relative to unconstrained peptide salt compositions.

Some embodiments provide a constrained salt of a peptide. Some embodiments provide a constrained salt of a peptide comprising a sequence selected from SEQ ID NO: 1-9, wherein the constrained salt comprises a phospholipid, a straight-chain fatty acid, a branched-chain fatty acid, or a combination of any of the foregoing, and wherein at least 90% of the peptide is in an alpha helical conformation. In some embodiments, the constraining salt is a straight-chain fatty acid. In some embodiments the constrained salt of the peptide is in solution.

Some embodiments provide a constrained salt of a peptide comprising SEQ ID NO: 1, wherein the salt comprises a phospholipid, a straight-chain fatty acid, a branched-chain fatty acid, or a combination of any of the foregoing, and wherein at least 90% of the peptide is in an alpha helical conformation. In some embodiments, the constraining salt is a straight-chain fatty acid. In some embodiments the constrained salt of the peptide is in solution.

Some embodiments provide a composition comprising, consisting or consisting essentially of about 0.001%-0.1% (e.g., 0.01% or 0.005%) of a constrained salt of a peptide, wherein the amount of peptide is weight/weight percentage, preferably calculated using the molecular weight of the free base and/or unmodified form of the peptide; about 0.03%-3% (e.g., 0.2888%) of a buffer; about 0.0001%-0.01% (e.g., 0.001%) disodium EDTA; optionally about 0.005%-0.5% (e.g., 0.05%) of a surfactant (e.g., tyloxapol, or n-DodecylPhosphoCholine (DPC)), and sodium chloride; wherein the pH of the composition is between about 6.2 to about 6.8 and the osmolality of the composition is between about 150-500 mOsm/kg or higher (e.g., 250 to 350) mOsm/kg. In some embodiments the composition comprises a solvent. In some embodiments the solvent is present in an amount of about 1%. In some embodiments the solvent is DMSO. In some embodiments, the composition is a liquid composition. Liquid compositions include, but are not limited to, combinations, mixtures, solutions, gel compositions and ointments.

In some embodiments, the buffer is phosphate buffered saline. In some embodiments, the buffer is a citrate buffer. In some embodiments, the citrate buffer comprises about 0.001%-0.1% (e.g., 0.0098%) anhydrous citric acid and about 0.02%-2% (e.g., 0.279%) sodium citrate dihydrate. In some embodiments, the pH of the composition is about 6.5.

In some embodiments, the osmolality of the composition is between about 280 to about 320 mOsm/kg. In some embodiments, the osmolality of the composition is about 300 mOsm/kg. In some embodiments, the amount of NaCl is between 0.4% and 0.6% (e.g., about 0.5%).

In some embodiments, the composition further comprises paraben such as methylparaben (e.g., 0.04% or less). In alternate embodiments, no parabens or other preservatives are included. In some embodiments, the composition further comprises sodium chlorite.

Some embodiments provide a kit, comprising a plurality of sterile single-use containers, wherein each container comprises a vessel for holding the composition. In some embodiments, the container comprises between about 0.03 mL to about 1 mL (e.g., 0.040 mL, 0.050 mL, 0.060 mL, 0.070 mL, 0.075 mL, 0.1 mL, 0.15 mL, 0.2 mL, 0.25 mL 0.3 mL, 0.35 mL, 0.4 mL, 0.45 mL, 0.5 mL, 0.6 mL, 0.7 mL, 0.8 mL, 0.9 mL) of the composition. In some embodiments, the container is for single use, daily use, weekly use or more long-term use, for example, a sterile blow fill seal container. In one embodiment, a 1 mL-30 mL container is used. The containers, in some embodiments are dropper bottles or gel/ointment tubes. In some embodiments, the container comprises a removable seal top for sealing the vessel, and a neck portion interconnecting the vessel and the seal top.

In some embodiments, the container is made from one or more of the following materials: polyvinyl chloride, polypropylene, polyethylene terephthalate, polyethylene terephthalate, polyethylene terephthalate G, high-density polyethylene, low-density polyethylene, polybutylene terephthalate, polyurethane, polyethylene vinyl acetate, silicone, acrylonitrile butadiene styrene, polytetrafluoroethylene, polycarbonate, polystyrene, polymethylmethacrylate, polysulfone, polyvinylidene chloride, or combinations thereof. Glass containers and surfaces that reduce the adhesion of the composition to the inner container surface are provided in some embodiments.

In some embodiments, the polypeptide is a constrained salt of Lacripep™ having SEQ ID NO: 1. In some embodiments, the polypeptide is a constrained salt having a sequence selected from the group of SEQ ID NOs: 2-9, or a fragment or fragments, thereof. In some embodiments, the constrained salt of the polypeptide has sequence homology of at least about 80%, 85% 90%, or 95% to SEQ ID NO: 1 or SEQ ID NOs: 2-9.

Some embodiments provide a method of administration comprising topically applying the composition to the eye, such as a sterile liquid eye drop from a single-use container. Some embodiments provide a method of administration comprising topically applying the composition to the eye, such as a sterile unpreserved liquid eye drop from a single-use container.

Some embodiments provide a use of the compositions described herein for the treatment of dry eye. Some embodiments provide a use of the compositions described herein for the treatment of one or more symptoms of Sjögren's Syndrome. In some embodiments, the composition comprises a constrained salt of Lacripep™.

In some embodiments of the peptide, constrained salt, composition, kit, method, or use, the protein or polypeptide is a constrained salt of the protein or polypeptide, wherein the salt comprises a phospholipid, a straight-chain fatty acid, a branched-chain fatty acid, or a combination of any of the foregoing. In some embodiments, the phospholipid, the straight-chain fatty acid, the branched-chain fatty acid, or the combination of any of the foregoing, is selected from salts of myristoleic acid, palmitoleic acid, sapienic acid, oleic acid, elaidic acid, vaccenic acid, linoleic acid, linolaidic acid, alpha-linolaidic acid, arachidonic acid, eicopentaenoic acid, eruric acid, docosahexaenoic acid, caprylic acid, capric acid, lauric acid, myristic acid, palmitic acid, stearic acid, arachidic acid, behenic acid, lignoceric acid, cerotic acid, phosphatidic acid, phosphatidylethanolamine, phosphatidylcholine (lecithin), dodecylphosphocholine, phosphatidylserine, phosphatidylinositol, phosphatidylinositol phosphate, phosphatidylinositol bisphosphate, phosphatidylinositol triphosphate, ceramide phosphorylcholine, ceramide phosphorylethanolamine, and combinations of any of the foregoing. In some embodiments, the constrained salt is a lineoleate salt. In some embodiments, the constrained salt is an oleic salt.

Some embodiments provide a constrained salt of a peptide comprising a sequence selected from SEQ ID NOs: 1-9, wherein the salt comprises a phospholipid, a straight-chain fatty acid, a branched-chain fatty acid, or a combination of any of the foregoing, and wherein at least 90% of the peptide is in an alpha helical conformation. Some embodiments provide a constrained salt of a peptide comprising SEQ ID NO: 1, wherein the salt comprises a phospholipid, a straight-chain fatty acid, a branched-chain fatty acid, or a combination of any of the foregoing, and wherein at least 90% of the peptide is in an alpha helical conformation. In some embodiments the salt is a straight-chain fatty acid.

Some embodiments provide a liquid composition comprising: 0.001-0.05% of a the constrained salt of a peptide disclosed above and herein, wherein the amount of peptide is weight/weight percentage, preferably calculated using the molecular weight of the free base and/or unmodified form of the peptide; 0.01-0.6% of a buffer; 0.0005-0.01% disodium EDTA; 0.01-0.1% tyloxapol, and sodium chloride; wherein the pH of the composition is between 6.2 to about 6.8 and the osmolality of the composition is between about 250 to 350 mOsm/kg, and wherein at least 90% of the peptide is in an alpha helical conformation. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. Some embodiments provide a liquid composition comprising: 0.001-0.05% of the constrained salt of a peptide disclosed above and herein, wherein the amount of peptide is weight/weight percentage, preferably calculated using the molecular weight of the free base and/or unmodified form of the peptide; 0.01-0.6% of a buffer; 0.0005-0.01% disodium EDTA; and sodium chloride; wherein the pH of the composition is between 6.2 to about 6.8 and the osmolality of the composition is between about 250 to 350 mOsm/kg, and wherein at least 90% of the peptide is in an alpha helical conformation. In some embodiments, the amount of the constrained salt of the peptide is 0.01% or 0.005%, wherein the amount of peptide is weight/weight percentage, preferably calculated using the molecular weight of the free base and/or unmodified form of the peptide; the buffer is 0.2888%; the disodium EDTA is 0.001%; the tyloxapol is 0.05%, the pH of the composition is between 6.2 to about 6.8 and the osmolality of the composition is between about 250 to 350 mOsm/kg; wherein the peptide is Lacripep having SEQ ID NO: 1, optionally wherein the N-terminus is acetylated and the C-terminus is amidated. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the buffer is a citrate buffer. In some embodiments the citrate buffer comprises 0.0098% anhydrous citric acid and 0.279% sodium citrate dihydrate. In some embodiments the pH of the composition is about 6.5. In some embodiments the osmolality of the composition is between 280 to 320 mOsm/kg. In some embodiments the osmolality of the composition is 300 mOsm/kg. In some embodiments the amount of NaCl is between 0.4% and 0.6%. In some embodiments the amount of NaCl is 0.5%. In some embodiments the composition further comprises 0.04% methylparaben. In some embodiments the composition is sterile.

Some embodiments provide a topical composition comprising: 0.005-0.05% of the constrained salt of a peptide disclosed above and herein, wherein the amount of peptide is weight/weight, preferably calculated using the molecular weight of the free base and/or unmodified form of the peptide, and one or more of the following: 0.1-0.6% of a buffer; 0.0005-0.01% disodium EDTA; 0.01-0.1% tyloxapol, and sodium chloride, wherein the composition is a solution, gel or ointment. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments the constrained salt of a peptide is provided in amount between 0.005% and 0.01%, wherein the amount of peptide is weight/weight percentage, preferably calculated using the molecular weight of the free base and/or unmodified form of the peptide. In some embodiments the constrained salt of the peptide is a tear glycoprotein or a fragment thereof. In some embodiments the constrained salt of the peptide comprises or consists of any one of SEQ ID NOs 1-9. In some embodiments the peptide is constrained Lacripep consisting of SEQ ID NO: 1, optionally wherein the N-terminus is acetylated and the C-terminus is amidated. In some embodiments, compositions disclosed above and herein are for use in treating one or more symptoms or signs of dry eye or Sjögren's Syndrome.

Some embodiments provide a kit, comprising a plurality of single-use containers, wherein each container comprises a vessel for holding a compositions disclosed above and herein. In some embodiments the container comprises between about 0.05 mL to about 1 mL of the composition. In some embodiments the container comprises a removable seal top for sealing the vessel, and a neck portion interconnecting the vessel and the seal top. In some embodiments the removable seal top cannot reseal the vessel once removed. In some embodiments the container comprises polyvinyl chloride, polypropylene, polyethylene terephthalate, polyethylene terephthalate, polyethylene terephthalate G, high-density polyethylene, low-density polyethylene, polybutylene terephthalate, polyurethane, polyethylene vinyl acetate, silicone, acrylonitrile butadiene styrene, polytetrafluoroethylene, polycarbonate, polystyrene, polymethylmethacrylate, polysulfone, polyvinylidene chloride, or combinations thereof.

Some embodiments provide a method of administration comprising topically applying one or more drops of a composition disclosed above and herein to the eye. In some embodiments the administration further comprises topically applying a drop of the composition to the eye from a single-use container of a kit disclosed above and herein.

Some embodiments provide a use of a composition disclosed above and herein for the treatment of dry eye. Some embodiments provide a use of a composition disclosed above and herein for the treatment of one or more symptoms or signs of Sjögren's Syndrome.

Some embodiments provide the constrained salt of a peptide, composition, kit, method, or use disclosed above and herein, wherein the salt comprises a phospholipid, a straight-chain fatty acid, a branched-chain fatty acid, or a combination of any of the foregoing. In some embodiments of the constrained salt of a peptide, composition, kit, method, or use the phospholipid, the straight-chain fatty acid, the branched-chain fatty acid, or the combination of any of the foregoing, is selected from salts of myristoleic acid, palmitoleic acid, sapienic acid, oleic acid, elaidic acid, vaccenic acid, linoleic acid, linolaidic acid, alpha-linolaidic acid, arachidonic acid, eicopentaenoic acid, eruric acid, docosahexaenoic acid, caprylic acid, capric acid, lauric acid, myristic acid, palmitic acid, stearic acid, arachidic acid, behenic acid, lignoceric acid, cerotic acid, phosphatidic acid, phosphatidylethanolamine, phosphatidylcholine (lecithin), dodecylphosphocholine, phosphatidylserine, phosphatidylinositol, phosphatidylinositol phosphate, phosphatidylinositol bisphosphate, phosphatidylinositol triphosphate, ceramide phosphorylcholine, ceramide phosphorylethanolamine, and combinations of any of the foregoing. In some embodiments, the constrained salt is a lineoleate salt. In some embodiments, the constrained salt is an oleic salt.

Some embodiments provide the constrained salt of a peptide, composition, kit, method, or use disclosed above and herein, wherein the peptide consists of SEQ ID NO:1, wherein the N-terminus is acetylated and the C-terminus is amidated. In some embodiments of the constrained salt of a peptide, composition, kit, method, or use, the amount of the peptide is about 0.01%, wherein the amount of peptide is weight/weight percentage, preferably calculated using the molecular weight of the free base and/or unmodified form of the peptide. In some embodiments of the constrained salt of a peptide, composition, kit, method, or use, the amount of the peptide is about 0.005%, wherein the amount of peptide is weight/weight percentage, preferably calculated using the molecular weight of the free base and/or unmodified form of the peptide. In some embodiments of the constrained salt of a peptide, composition, kit, method, or use, the tyloxapol is replaced with a surfactant. In some embodiments the constrained salt of the a peptide, composition, kit, method, or use, the surfactant comprises or consists of DPC. In some embodiments the constrained salt of a peptide, composition, kit, method, or use, comprises a solvent. In some embodiments the solvent is present in an amount of about 0.1-10%. In some embodiments the solvent is present in an amount of about 1%. In some embodiments the solvent is DMSO.

In some embodiments, the peptide (or combination of peptides) is constrained in the compositions of the present application so as to allow for long-term storage while maintaining efficacy and/or delivery over a prolonged period of time. As such, in some embodiments these constrained peptide compositions, and pharmaceutically acceptable salts thereof, are stable at non-refrigerated temperatures without the need for reconstitution, and are functional over a range of temperatures, including temperatures ranging from 0-40° C. In some embodiments, these constrained peptide compositions, and pharmaceutically acceptable salts thereof, also provide increased chemical and/or biological stability and/or additional efficacy relative to unconstrained peptide compositions.

Some embodiments provide a peptide comprising SEQ ID NO: 1, or a pharmaceutically acceptable salt thereof, wherein at least two amino acids at the i and i+3, i and i+4, or i and i+7 positions of a helical turn are replaced or modified with a compound comprising a crosslinking moiety and said crosslinking moieties are covalently bonded at the i and i+3, the i and i+4 or at the i and i+7 positions, and wherein said peptide has an alpha helical conformation.

Some embodiments provide a peptide comprising SEQ ID NO: 2, or a pharmaceutically acceptable salt thereof, wherein at least two amino acids at the i and i+3, i and i+4, or i and i+7 positions of a helical turn are replaced or modified with a compound comprising a crosslinking moiety and said crosslinking moieties are covalently bonded, and wherein said peptide has an alpha helical conformation.

In some embodiments, the two amino acids at the i and i+3 position are or modified with a compound comprising a crosslinking moiety and said crosslinking moieties are covalently bonded. In some embodiments, the two amino acids at the i and i+4 position are or modified with a compound comprising a crosslinking moiety and said crosslinking moieties are covalently bonded. In some embodiments, the two amino acids at the i and i+7 position are replaced or modified with a compound comprising a crosslinking moiety and said crosslinking moieties are covalently bonded.

In some embodiments, the compound comprising a crosslinking moiety comprises a compound of Formula (I).

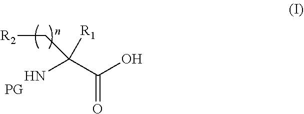

In some embodiments, each n is independently 0, 1, 2, 3, 4, 5, 6, 7, 8, 9, or 10. In some embodiments, each PG is independently Boc or Fmoc. In some embodiments, each R₁ is independently hydrogen or methyl. In some embodiments, each R₂ is independently selected from the group consisting of:

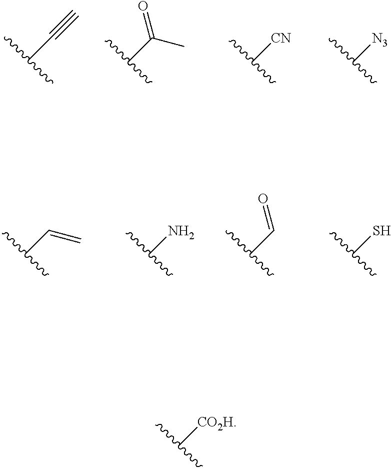

In some embodiments, each n is independently 3, 4, 5, 6, or 7. In some embodiments, each PG is Fmoc. In some embodiments, each R₁ is hydrogen. In some embodiments, each R² is

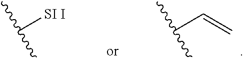

Some embodiments provide a method of constraining Lacripep, comprising contacting a peptide having SEQ ID NO: 1, wherein at least two amino acids are replaced or modified with compounds comprising a crosslinking moiety are contacted with a reagent that is an oxidizing agent, a transition metal catalyst or an olefin methathesis catalyst; when the crosslinking moieties are each an alkene or an azide and an alkyne, the reagent is a transition metal catalyst; when the crosslinking moieties are each a sulfhydryl, the reagent is an oxidizing agent; when the crosslinking moieties are each an alkene, the reagent is an olefin metathesis catalyst; and when the crosslinking moieties are crosslinked, at least 90% of the peptide is in an alpha helical conformation.

Some embodiments provide a composition comprising, consisting or consisting essentially of about 0.001%-0.1% (e.g., 0.01% or 0.005%) of a constrained peptide (or combination of constrained peptides), or a pharmaceutically acceptable salt thereof, wherein the amount of peptide is weight/weight percentage calculated using the molecular weight of the free base form of the peptide; about 0.03%-3% (e.g., 0.2888%) of a buffer; about 0.0001%-0.01% (e.g., 0.001%) disodium EDTA; optionally about 0.005%-0.5% (e.g., 0.05%) of a surfactant (e.g., tyloxapol, or n-DodecylPhosphoCholine (DPC)) and sodium chloride; wherein the pH of the composition is between about 6.2 to about 6.8 and the osmolality of the composition is between about 150-500 mOsm/kg or higher (e.g., 250 to 350) mOsm/kg. In some embodiments, the composition is a liquid composition. Liquid compositions include, but are not limited to, combinations, mixtures, solutions, gel compositions and ointments.

In some embodiments, the buffer is phosphate buffered saline. In some embodiments, the buffer is a citrate buffer. In some embodiments, the citrate buffer comprises about 0.001%-0.1% (e.g., 0.0098%) anhydrous citric acid and about 0.02%-2% (e.g., 0.279%) sodium citrate dihydrate. In some embodiments, the pH of the composition is about 6.5.

In some embodiments, the osmolality of the composition is between about 280 to about 320 mOsm/kg. In some embodiments, the osmolality of the composition is about 300 mOsm/kg. In some embodiments, the amount of NaCl is between 0.4% and 0.6% (e.g., about 0.5%).

In some embodiments, the composition further comprises paraben such as methylparaben (e.g., 0.04% or less). In alternate embodiments, no parabens or other preservatives are included. In some embodiments, the composition further comprises sodium chlorite.

Some embodiments provide a kit, comprising a plurality of sterile single-use containers, wherein each container comprises a vessel for holding the composition. In some embodiments, the container comprises between about 0.03 mL to about 1 mL (e.g., 0.040 mL, 0.050 mL, 0.060 mL, 0.070 mL, 0.075 mL, 0.1 mL, 0.15 mL, 0.2 mL, 0.25 mL 0.3 mL, 0.35 mL, 0.4 mL, 0.45 mL, 0.5 mL, 0.6 mL, 0.7 mL, 0.8 mL, 0.9 mL) of the composition. In some embodiments, the container is for single use, daily use, weekly use or more long-term use, for example, a sterile blow fill seal container. In one embodiment, a 1 mL-30 mL container is used. The containers, in some embodiments are dropper bottles or gel/ointment tubes. In some embodiments, the container comprises a removable seal top for sealing the vessel, and a neck portion interconnecting the vessel and the seal top.

In some embodiments, the container is made from one or more of the following materials: polyvinyl chloride, polypropylene, polyethylene terephthalate, polyethylene terephthalate, polyethylene terephthalate G, high-density polyethylene, low-density polyethylene, polybutylene terephthalate, polyurethane, polyethylene vinyl acetate, silicone, acrylonitrile butadiene styrene, polytetrafluoroethylene, polycarbonate, polystyrene, polymethylmethacrylate, polysulfone, polyvinylidene chloride, or combinations thereof. Glass containers and surfaces that reduce the adhesion of the composition to the inner container surface are provided in some embodiments.

In some embodiments, the peptide is constrained Lacripep™ having SEQ ID NO: 1, or a pharmaceutically acceptable salt thereof, wherein at least two amino acids of Lacripep™ are replaced or modified to comprise a crosslinking moiety, as described herein. In some embodiments, the constrained peptide has a sequence selected from the group of SEQ ID NOs: 2-9, or pharmaceutically acceptable salt, or a fragment or fragments, thereof, wherein at least two amino acids of the sequence are replaced or modified to comprise a crosslinking moiety, as described herein. In some embodiments, the constrained peptide, or a pharmaceutically acceptable salt thereof, has sequence homology of at least about 80%, 85% 90%, 95%, or 98% to SEQ ID NO: 1 or SEQ ID NOs: 2-9.

Some embodiments provide a method of administration comprising topically applying the composition to the eye, such as a sterile liquid eye drop from a single-use container. Some embodiments provide a method of administration comprising topically applying the composition to the eye, such as a sterile unpreserved liquid eye drop from a single-use container.

Some embodiments provide a use of the compositions described herein for the treatment of dry eye. Some embodiments provide a use of the compositions described herein for the treatment of one or more symptoms of Sjögren's Syndrome. In some embodiments, the composition comprises constrained Lacripep™, or a pharmaceutically acceptable salt thereof.

Some embodiments provide a peptide comprising SEQ ID NO: 1, or a pharmaceutically acceptable salt thereof, wherein at least two amino acids at the i and i+4 or i and i+7 positions of a helical turn are replaced or modified with a compound comprising a crosslinking moiety and said crosslinking moieties are covalently bonded, and wherein said peptide has an alpha helical conformation. Some embodiments provide a peptide comprising SEQ ID NO: 2, or a pharmaceutically acceptable salt thereof, wherein at least two amino acids at the i and i+4 or i and i+7 positions of a helical turn are replaced or modified with a compound comprising a crosslinking moiety and said crosslinking moieties are covalently bonded, and wherein said peptide has an alpha helical conformation. In some embodiments the two amino acids at the i and i+4 position are or modified with a compound comprising a crosslinking moiety and said crosslinking moieties are covalently bonded. In some embodiments the two amino acids at the i and i+7 position are replaced or modified with a compound comprising a crosslinking moiety and said crosslinking moieties are covalently bonded. In some embodiments the compound comprising a crosslinking moiety comprises a compound of Formula (I):

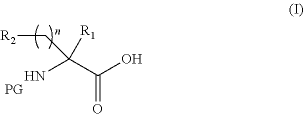

wherein: each n is independently 0, 1, 2, 3, 4, 5, 6, 7, 8, 9, or 10; each PG is independently Boc or Fmoc; each R₁ is independently hydrogen or methyl; and each R₂ is independently selected from the group consisting of:

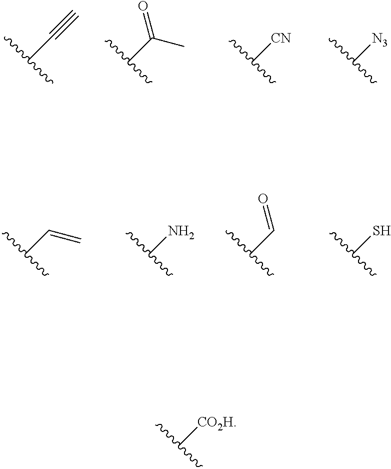

In some embodiments each n is independently 3, 4, 5, 6, or 7. In some embodiments each PG is Fmoc. In some embodiments each R₁ is hydrogen. In some embodiments each R² is

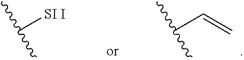

Some embodiments provide a liquid composition comprising: 0.001-0.05% of a constrained peptide, or a pharmaceutically acceptable salt thereof, disclosed above and herein, wherein the amount of peptide is weight/weight percentage, preferably calculated using the molecular weight of the free base and/or unconstrained form of the peptide; 0.01-0.6% of a buffer; 0.0005-0.01% disodium EDTA; 0.01-0.1% tyloxapol, and sodium chloride; wherein the pH of the composition is between 6.2 to about 6.8 and the osmolality of the composition is between about 250 to 350 mOsm/kg, and wherein at least 90% of the peptide is in an alpha helical conformation. In some embodiments the composition comprises DPC, and/or another surfactant instead of or in addition to tyloxapol. Some embodiments provide a liquid composition comprising: 0.001-0.05% of a constrained peptide, or a pharmaceutically acceptable salt thereof, disclosed above and herein, wherein the amount of peptide is weight/weight percentage, preferably calculated using the molecular weight of the free base and/or unconstrained form of the peptide; 0.01-0.6% of a buffer; 0.0005-0.01% disodium EDTA; and sodium chloride; wherein the pH of the composition is between 6.2 to about 6.8 and the osmolality of the composition is between about 250 to 350 mOsm/kg, and wherein at least 90% of the peptide is in an alpha helical conformation. In some embodiments the amount of the constrained peptide, or a pharmaceutically acceptable salt thereof, is 0.01% or 0.005%, wherein the amount of peptide is weight/weight percentage calculated using the molecular weight of the free base and/or unconstrained form of the peptide; wherein the buffer is 0.2888%; wherein the disodium EDTA is 0.001%; wherein the tyloxapol is 0.05%, wherein the pH of the composition is between 6.2 to about 6.8 and the osmolality of the composition is between about 250 to 350 mOsm/kg; wherein the peptide is constrained Lacripep having SEQ ID NO: 1, or a pharmaceutically acceptable salt thereof. In some embodiments the composition comprises DPC, and/or another surfactant instead of or in addition to tyloxapol. In some embodiments the buffer is a citrate buffer. In some embodiments the citrate buffer comprises 0.0098% anhydrous citric acid and 0.279% sodium citrate dihydrate. In some embodiments the pH of the composition is about 6.5. In some embodiments the osmolality of the composition is between 280 to 320 mOsm/kg. In some embodiments the osmolality of the composition is 300 mOsm/kg. In some embodiments the amount of NaCl is between 0.4% and 0.6%. In some embodiments the amount of NaCl is 0.5%. In some embodiments the composition further comprises 0.04% methylparaben.

Some embodiments provide a topical composition comprising: 0.005-0.05% of the constrained peptide disclosed above and herein, wherein the amount of peptide is weight/weight percentage, preferably calculated using the molecular weight of the free base and/or unconstrained form of the peptide and one or more of the following: 0.1-0.6% of a buffer; 0.0005-0.01% disodium EDTA; 0.01-0.1% tyloxapol, and sodium chloride, wherein the composition is a solution, gel or ointment. In some embodiments the composition comprises DPC, and/or another surfactant instead of or in addition to tyloxapol. In some embodiments the amount of the constrained peptide is between 0.001 and 0.01%, wherein the amount of peptide is weight/weight percentage, preferably calculated using the molecular weight of the free base and/or unconstrained form of the peptide. In some embodiments the constrained peptide is a tear glycoprotein or a fragment thereof. In some embodiments the constrained peptide is any one of SEQ ID NOs 1-9, wherein at least two of the amino acids comprise a crosslinking moiety. In some embodiments the peptide is constrained Lacripep. In some embodiments the composition is sterile.

Some embodiments provide a kit, comprising a plurality of single-use containers, wherein each container comprises a vessel for holding a composition disclosed above and herein. In some embodiments the container comprises between about 0.05 mL to about 1 mL of the composition. In some embodiments the container comprises a removable seal top for sealing the vessel, and a neck portion interconnecting the vessel and the seal top. In some embodiments the removable seal top cannot reseal the vessel once removed. In some embodiments the container comprises polyvinyl chloride, polypropylene, polyethylene terephthalate, polyethylene terephthalate, polyethylene terephthalate G, high-density polyethylene, low-density polyethylene, polybutylene terephthalate, polyurethane, polyethylene vinyl acetate, silicone, acrylonitrile butadiene styrene, polytetrafluoroethylene, polycarbonate, polystyrene, polymethylmethacrylate, polysulfone, polyvinylidene chloride, or combinations thereof.

Some embodiments provide a method of administration comprising topically applying one or more drops of a composition disclosed above and herein to the eye. In some embodiments the administration further comprises topically applying a drop of the composition to the eye from a single-use container of a kit disclosed above and herein.

Some embodiments provide a use of a composition disclosed above and herein for the treatment of dry eye. Some embodiments provide a use of a composition disclosed above and herein for the treatment of one or more symptoms or signs of Sjögren's Syndrome.

Some embodiments provide a method of constraining Lacripep, comprising contacting a peptide having SEQ ID NO: 1 with a crosslinking reagent, wherein: at least two amino acids are replaced or modified with compounds comprising a crosslinking moiety are contacted with a reagent selected from an oxidizing agent and a transition metal catalyst; when the crosslinking moieties are each an alkene or an azide and an alkyne, the reagent is a transition metal catalyst; when the crosslinking moieties are each a sulfhydryl, the reagent is an oxidizing agent; when the crosslinking moieties are each an alkene, the reagent is an olefin metathesis catalyst; and when the crosslinking moieties are crosslinked, at least 90% of the peptide is in an alpha helical conformation.

Some embodiments provide a constrained peptide, composition, kit, method, or use disclosed above and herein, wherein the peptide consists of a constrained form of SEQ ID NO:1, wherein the N-terminus is acetylated and the C-terminus is amidated. In some embodiments the constrained peptide, composition, kit, method, or use, the amount of the peptide is about 0.01%, wherein the amount of peptide is weight/weight percentage, preferably calculated using the molecular weight of the free base and/or unconstrained form of the peptide. In some embodiments the constrained peptide, composition, kit, method, or use, the amount of the peptide is about 0.005%, wherein the amount of peptide is weight/weight percentage, preferably calculated using the molecular weight of the free base and/or unconstrained form of the peptide. In some embodiments the constrained peptide, composition, kit, method, or use, the tyloxapol is replaced with a surfactant. In some embodiments the constrained peptide, composition, kit, method, or use, the surfactant comprises or consists of DPC.

In some embodiments, provided is a constrained salt of a peptide comprising a sequence selected from SEQ ID NOs: 1-9, wherein the salt comprises a phospholipid, a straight-chain fatty acid, a branched-chain fatty acid, or a combination of any of the foregoing, and wherein at least 90% of the peptide is in an alpha helical conformation. In some embodiments, provided is a constrained salt of a peptide comprising SEQ ID NO: 1, wherein the salt comprises a phospholipid, a straight-chain fatty acid, a branched-chain fatty acid, or a combination of any of the foregoing, and wherein at least 90% of the peptide is in an alpha helical conformation. In some embodiments, the salt is a straight-chain fatty acid. In some embodiments, provided is a peptide comprising SEQ ID NO: 1, or a pharmaceutically acceptable salt thereof, wherein at least two amino acids at the i and i+4 or i and i+7 positions of a helical turn are replaced or modified with a compound comprising a crosslinking moiety and said crosslinking moieties are covalently bonded, and wherein said peptide has an alpha helical conformation. In some embodiments, provided is a peptide comprising SEQ ID NO: 2, or a pharmaceutically acceptable salt thereof, wherein at least two amino acids at the i and i+4 or i and i+7 positions of a helical turn are replaced or modified with a compound comprising a crosslinking moiety and said crosslinking moieties are covalently bonded, and wherein said peptide has an alpha helical conformation. In some embodiments, the two amino acids at the i and i+4 position are or modified with a compound comprising a crosslinking moiety and said crosslinking moieties are covalently bonded. In some embodiments, the two amino acids at the i and i+7 position are replaced or modified with a compound comprising a crosslinking moiety and said crosslinking moieties are covalently bonded. In some embodiments, the compound comprising a crosslinking moiety comprises a compound of Formula (I):

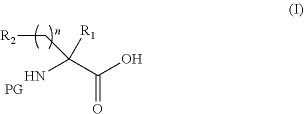

wherein: each n is independently 0, 1, 2, 3, 4, 5, 6, 7, 8, 9, or 10; each PG is independently Boc or Fmoc; each R₁ is independently hydrogen or methyl; and each R₂ is independently selected from the group consisting of:

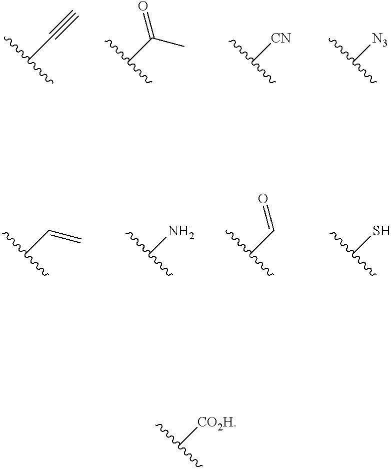

The peptide of claim 8, wherein each n is independently 3, 4, 5, 6, or 7. In some embodiments, each PG is Fmoc. In some embodiments, each R₁ is hydrogen. In some embodiments, each R₂ is or

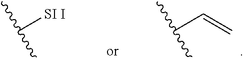

In some embodiments, provided is a liquid composition comprising: 0.00001-0.05% or 0.0001-0.05% of the constrained salt of a peptide or constrained peptide of any of the above embodiments, wherein the amount of peptide is weight/weight percentage calculated using the molecular weight of the free base and/or unconstrained form of the peptide; 0.01-0.6% of a buffer; 0.0005-0.01% disodium EDTA; and sodium chloride; wherein the pH of the composition is between 6.2 to about 6.8 and the osmolality of the composition is between about 150 to 350 mOsm/kg, and wherein at least 90% of the peptide is in an alpha helical conformation. In some embodiments, provided is a topical composition comprising: 0.00001-0.05% of the constrained salt of a peptide or constrained peptide of any of the above embodiments, wherein the amount of peptide is weight/weight percentage calculated using the molecular weight of the free base and/or unconstrained form of the peptide, and one or more of the following: 0.1-0.6% of a buffer; 0.0005-0.01% disodium EDTA; and sodium chloride, wherein the composition is a solution, gel or ointment. In some embodiments, the composition further comprises 0.01-0.1% tyloxapol. In some embodiments, the amount of the constrained salt of the peptide or constrained peptide is 0.01%, 0.005%, 0.001%, 0.0001% or 0.00001%, wherein the amount of peptide is weight/weight percentage calculated using the molecular weight of the free base and/or unconstrained form of the peptide; wherein the buffer is 0.2888%; wherein the disodium EDTA is 0.001%; wherein the tyloxapol is 0.05%, wherein the pH of the composition is between 6.2 to about 6.8 and the osmolality of the composition is between about 150 to 350 mOsm/kg; wherein the peptide is Lacripep having SEQ ID NO: 1, optionally wherein the N-terminus is acetylated and the C-terminus is amidated. In some embodiments, the buffer is a citrate buffer. In some embodiments, the citrate buffer comprises 0.0098% anhydrous citric acid and 0.279% sodium citrate dihydrate. In some embodiments, the pH of the composition is about 6.5. In some embodiments, the osmolality of the composition is between 150 to 250 mOsm/kg. In some embodiments, the osmolality of the composition is about 200 mOsm/kg. In some embodiments, the amount of NaCl is between 0.4% and 0.6%. In some embodiments, the amount of NaCl is 0.5%. In some embodiments, composition further comprises 0.04% methylparaben. In some embodiments, the composition is sterile. In some embodiments, the peptide is constrained Lacripep or a constrained salt of Lacripep consisting of SEQ ID NO: 1, optionally wherein the N-terminus is acetylated and the C-terminus is amidated. In some embodiments, the composition is for use in treating one or more symptoms or signs of dry eye or Sjögren's Syndrome. In some embodiments, provided is a kit, comprising a plurality of single-use containers, wherein each container comprises a vessel for holding the composition of of any of the above embodiments. In some embodiments, the container comprises between about 0.05 mL to about 1 mL of the composition. In some embodiments, the container comprises a removable seal top for sealing the vessel, and a neck portion interconnecting the vessel and the seal top. In some embodiments, the removable seal top cannot reseal the vessel once removed. In some embodiments, the container comprises polyvinyl chloride, polypropylene, polyethylene terephthalate, polyethylene terephthalate, polyethylene terephthalate G, high-density polyethylene, low-density polyethylene, polybutylene terephthalate, polyurethane, polyethylene vinyl acetate, silicone, acrylonitrile butadiene styrene, polytetrafluoroethylene, polycarbonate, polystyrene, polymethylmethacrylate, polysulfone, polyvinylidene chloride, or combinations thereof. In some embodiments, provided is a method of administration comprising topically applying one or more drops of the composition of any of the above embodiments. In some embodiments, the administration further comprises topically applying a drop of the composition to the eye from a single-use container of the kit of any of the above embodiments. In some embodiments, provided is a use of the composition of any of the above embodiments for the treatment of dry eye. In some embodiments, provided is a use of the composition of any of the above embodiments for the treatment of one or more symptoms or signs of Sjögren's Syndrome. In some embodiments, the salt comprises a phospholipid, a straight-chain fatty acid, a branched-chain fatty acid, or a combination of any of the foregoing. In some embodiments, the phospholipid, the straight-chain fatty acid, the branched-chain fatty acid, or the combination of any of the foregoing, is selected from salts of myristoleic acid, palmitoleic acid, sapienic acid, oleic acid, elaidic acid, vaccenic acid, linoleic acid, linolaidic acid, alpha-linolaidic acid, arachidonic acid, eicopentaenoic acid, eruric acid, docosahexaenoic acid, caprylic acid, capric acid, lauric acid, myristic acid, palmitic acid, stearic acid, arachidic acid, behenic acid, lignoceric acid, cerotic acid, phosphatidic acid, phosphatidylethanolamine, phosphatidylcholine (lecithin), dodecylphosphocholine, phosphatidylserine, phosphatidylinositol, phosphatidylinositol phosphate, phosphatidylinositol bisphosphate, phosphatidylinositol triphosphate, ceramide phosphorylcholine, ceramide phosphorylethanolamine, and combinations of any of the foregoing. In some embodiments, the constrained salt is a lineoleate salt. In some embodiments, the constrained salt is an oleic salt. In some embodiments, provided is a method of constraining Lacripep, comprising contacting a peptide having SEQ ID NO: 1 with a crosslinking reagent, wherein: at least two amino acids are replaced or modified with compounds comprising a crosslinking moiety are contacted with a reagent selected from an oxidizing agent and a transition metal catalyst; when the crosslinking moieties are each an alkene or an azide and an alkyne, the reagent is a transition metal catalyst; when the crosslinking moieties are each a sulfhydryl, the reagent is an oxidizing agent; and when the crosslinking moieties are crosslinked, at least 90% of the peptide is in an alpha helical conformation. In some embodiments, the peptide consists of SEQ ID NO:1, wherein the N-terminus is acetylated and the C-terminus is amidated. In some embodiments, the amount of the peptide is about 0.01%, wherein the amount of peptide is weight/weight percentage calculated using the molecular weight of the free base and/or unconstrained form of the peptide. In some embodiments, the amount of the peptide is about 0.005%, wherein the amount of peptide is weight/weight percentage calculated using the molecular weight of the free base and/or unconstrained form of the peptide. In some embodiments, the amount of the peptide is about 0.0001%, wherein the amount of peptide is weight/weight percentage calculated using the molecular weight of the free base and/or unconstrained form of the peptide. In some embodiments, the amount of the peptide is about 0.00001%, wherein the amount of peptide is weight/weight percentage calculated using the molecular weight of the free base and/or unconstrained form of the peptide. In some embodiments, the tyloxapol is replaced with a surfactant. In some embodiments, the surfactant comprises or consists of DPC. In some embodiments, the constrained peptide, constrained salt of a peptide, composition, kit, method, or use of any of the above embodiments further comprises a solvent. In some embodiments, the solvent is present in an amount of about 0.1 to about 10%. In some embodiments, the solvent is present in an amount of about 1%. In some embodiments, the solvent is DMSO.

### BRIEF DESCRIPTION OF THE DRAWINGS

FIG. 1 shows the results of the CD spectra of embodiments of constrained salts of a peptide.

FIG. 2 shows the results of the CD spectra of additional constrained salts of a peptide.

FIG. 3 shows the results of the CD spectra of embodiments of stapled lacripep peptides in the presence of 10 mM DPC.

FIG. 4 shows the results of the CD spectra of embodiments of stapled lacripep peptides in the absence of 10 mM DPC.

FIG. 5 shows the results of the CD spectra of embodiments of a cysteine disulfide-bridged lacripep peptides in the presence of DPC.

FIG. 6 shows the results of the CD spectra of embodiments of a cysteine disulfide-bridged lacripep peptides in the absence of 10 mM DPC.

### DETAILED DESCRIPTION

The peptides disclosed in the present application form random coils in solution, but under certain conditions can be in an alpha-helical conformation. In order to diminish the random coil and enhance the helical structural features of Lacripep™ and the other peptides disclosed in the present application, some embodiments of the present application describe constrained salts of peptides, and some embodiments of the present application describe constrained peptides and pharmaceutically acceptable salts thereof.

The identification of residues within the helical structure of Lacripep™, or the other peptides described herein, can be aided by the representation of their sequences in a helical wheel. For example, the helical wheel of Lacripep™ is shown below.

This representation demonstrates the amphipathic nature of the Lacripep™ sequence where lipophilic or hydrophobic residues and hydrophilic residues are segregated into two separate faces of the peptide and places residues i, i+3, i+4, and i+7 in sufficient proximity to be covalently linked or conformationally stabilized by association with a lipophilic or hydrophobic surface formed by the self-associating stabilizing salt. Although any set of amino acid sidechains which are close in space can be chemically linked to stabilize the turn of a helix, it may be advantageous to make connections along either the lipophilic/hydrophobic or hydrophilic face of the helix and not between the two faces.

Stabilizing the helical structure of Lacripep™, and the other peptides described in the present application, can provide benefits including one or more of improved biological potency in the treatment of dry eye, improved pharmacokinetic lifetime in tear, at the ocular surface and in periocular tissue, and improved chemical stability in formulation.

The following are illustrative definitions of terms used herein. Unless expressly stated otherwise, all technical and scientific terms used herein have the same meaning as is commonly understood by one of ordinary skill in the art read in light of the entire specification. All patents, applications, published applications and other publications referenced herein are incorporated by reference in their entirety unless stated otherwise.

The term “about,” as used herein, refers to a quantity, value, number, frequency, percentage, amount, or weight that varies +/−10% to a reference quantity, value, number, frequency, percentage, amount, or weight.

Unless indicated otherwise, when a percentage (%) value is used in the present application, the value refers to a weight/weight percent value. For the percentage (% w/w) value of peptides, constrained peptides, constrained salts of peptides, and pharmaceutically acceptable salts thereof, disclosed herein, the percentage (% w/w) is calculated using the molecular weight of the free base form of the unmodified or unconstrained peptide. Thus, the percentage (% w/w) will need to be adjusted when a salt form and/or modified form of the peptide is used if the same molar concentration of the peptide in solution is desired, as is the case in some embodiments herein.

For example, constrained salt forms of Lacripep™ as disclosed herein have a higher molecular weight than the free base form of Lacripep™, and therefore would need to be used in an amount greater than 0.01% (w/w), for example, to have the same molar concentration of Lacripep™ as a composition containing 0.01% (w/w) of free base Lacripep™. Non-limiting examples of the molecular weights of tetra-fatty acid salt forms of Lacripep™ in comparison to free base Lacripep™ are disclosed in the following table:

TM
tetra-Fatty

| | | Lacripep ™ tetra- |
|---|---|---|
| | | Fatty Acid Salt, |
| Fatty Acid | MW, amu | calculated MW, amu |
| None (Lacripep ™ | 2283.7 | NA |
| free base) | | |
| Lauric | 200.32 | 3084.98 |
| Myristic | 228.37 | 3197.18 |
| Palmitic | 256.43 | 3309.42 |
| Stearic | 284.48 | 3421.62 |
| Oleic | 282.47 | 3413.58 |
| Linoleic | 280.45 | 3405.5 |

The term “tonicity agent” as used herein includes materials that alter the osmolality of a composition. Suitable tonicity agents include, but are not limited to, propylene glycol, polyethylene glycols, sodium chloride, potassium chloride, magnesium chloride, calcium chloride, simple sugars such as dextrose, fructose, galactose, and/or simple polyols such as the sugar alcohols mannitol, sorbitol, xylitol, lactitol, isomaltitol, maltitol, hydrogenated starch hydrolysates, glycerin, and combinations of the foregoing.

The term “stabilizing agent” as used herein includes a material that inhibits chemical reactions with a peptide. Stabilizing agents may include, for example, antioxidants such as sodium metabisulfite, sodium thiosulfate, acetylcysteine, butylated hydroxyanisole, butylated hydroxytoluene, and combinations of the foregoing.

The term “surfactant” as used herein includes amphiphilic molecules, meaning that they contain both hydrophobic groups (tails) and hydrophilic groups (heads). Therefore, a surfactant contains both a water insoluble (or oil soluble) component and a water-soluble component. As used herein, surfactants may be detergents, wetting agents, emulsifiers, foaming agents, or dispersants. In some embodiments, the constrained salt of a polypeptide, or the constrained polypeptide, can act as a surfactant.

The term “chelating agent,” as used herein includes a compound that can form two or more bonds to a metal ion, i.e., a multi-dentate ligand. Chelating agents include, but are not limited to ethylenediaminetetraacetic acid (EDTA), ethylenediamine, amino acids such as glutamic acid and histidine, organic diacids such as oxalic acid, malonic acid, succinic acid, and the like, and pharmaceutically acceptable salts of the foregoing. In several embodiments, a chelating agent is EDTA, or a pharmaceutically acceptable salt thereof. In some embodiments, the constrained salt of a polypeptide, or constrained polypeptide, or pharmaceutically acceptable salt thereof, can act as a chelator.

The term “viscosity building agent” as used herein, includes materials that affect the viscosity (centipoise, or Cp) of a composition. Examples of viscosity enhancing agents include, but are not limited to: polysaccharides, such as hyaluronic acid and its salts, chondroitin sulfate and its salts, dextrans, various polymers of the cellulose family (and derivatives thereof), vinyl polymers, and acrylic acid polymers. Non-limiting examples of viscosity building agents include polyvinylalcohol (PVA), polyvinylpyrrolidone (PVP), polyethylene glycol (PEG), and polyacrylic acid (PAA).

The term “ophthalmically acceptable” as used herein includes materials that are compatible with ocular tissue; that is, it does not cause significant or undue detrimental effects when brought into contact with ocular tissue.

The terms “stable,” “stability” or “stabilized” as used herein includes products and compositions that enhance the primary, secondary and/or tertiary structure of the polypeptide, but without forming covalent bonds or salts with the polypeptide. In some embodiments, stabilized compositions may have an acceptable percentage of peptide degradation, or aggregation, products after a given period of time. These peptide degradation products can be the result of, for example, oxidation and/or hydrolysis of the peptide.

The terms “peptide”, “polypeptide” and “protein” as used herein, are used interchangeably. Unless otherwise clear from the context, the noted terms include a polymer having at least two amino acids linked through peptide bonds. The terms thus include oligopeptides, analogs, derivatives, acetylated derivatives, glycosylated derivatives, pegylated derivatives, and the like.

The terms “constrained peptide”, “constrained polypeptide” and “constrained protein” as used herein, are used interchangeably, and include a polymer having natural and/or non-natural amino acids wherein at least two amino acids comprise moieties capable of undergoing a reaction(s) to form a covalent bond (become “crosslinked”) between the amino acids in addition to the peptide backbone (hereinafter, “crosslinking moieties”) that promotes a helical conformation of the polymer (e.g. peptide), preferably an alpha helix. In some embodiments, the covalent bond comprises a carbon-carbon bond, a carbon-nitrogen bond, a carbon-oxygen bond, an amide bond, a disulfide bond, or a cycloaddition. When the crosslinking moieties are crosslinked, that is, undergo a reaction to form a covalent bond that promotes a helical conformation, this crosslinking is referred to as “constraining” a peptide or combination of peptides.

The term “constraining salt,” or “constrained salt” refers to a salt, preferably a pharmaceutically acceptable salt, that self-associates to promote a helical conformation of a peptide, polymer or polypeptide, preferably an alpha helix conformation. In some embodiments the constrained salt is in solution. Constraining salts include compounds that will self-associate to form a micelle or lipid bilayer. Examples of constraining salts include, but are not limited to salts of straight chain fatty acids, branched chain fatty acids, phospholipids and combinations of any of the foregoing. Non-limiting examples of constraining salts include salts of myristoleic acid, palmitoleic acid, sapienic acid, oleic acid, elaidic acid, vaccenic acid, linoleic acid, linolaidic acid, alpha-linolaidic acid, arachidonic acid, eicopentaenoic acid, eruric acid, docosahexaenoic acid, caprylic acid, capric acid, lauric acid, myristic acid, palmitic acid, stearic acid, arachidic acid, behenic acid, lignoceric acid, cerotic acid, phosphatidic acid, phosphatidylethanolamine, phosphatidylcholine (lecithin), phosphatidylserine, phosphatidylinositol, phosphatidylinositol phosphate, phosphatidylinositol bisphosphate, phosphatidylinositol triphosphate, ceramide phosphorylcholine, ceramide phosphorylethanolamine, and combinations of any of the foregoing. In some embodiments, the constrained salt is a lineoleate salt, which has a strong signal in the 220-230 nm range in a circular dichromism analysis, which is consistent with helical structure of this salt. In some embodiments, the constrained salt is an oleic salt. In some embodiments the constraining salt associates with the lipophilic or hydrophobic face of amphipathic helix, thereby stabilizing the helical structure. In some embodiments the constraining salt does not form a salt bridge between amino acids in the peptide. In some embodiments the constraining salt is a salt dodecylphosphocholine. In some embodiments the constraining salt is a mixture of salts.

The term “amino acid” refers to a molecule containing both an amino group and a carboxyl group. Amino acids include alpha-amino acids and beta-amino acids. In some embodiments, an amino acid is an alpha amino acid. In some embodiments, the amino acid is a natural amino acid. In some embodiments, the amino acid is an unnatural amino acid. In some embodiments, the unnatural amino acid comprises a (D) amino acid or a (L) amino acid. In some embodiments, the unnatural amino acid comprises a compound of Formula (I).

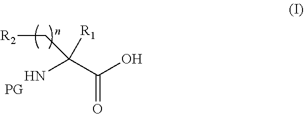

In some embodiments, each n is independently 0, 1, 2, 3, 4, 5, 6, 7, 8, 9, or 10. In some embodiments, PG is a suitable amino-protecting group. In some embodiments, each R₁ is independently hydrogen or methyl. In some embodiments, each R₂ is independently selected from the group consisting of:

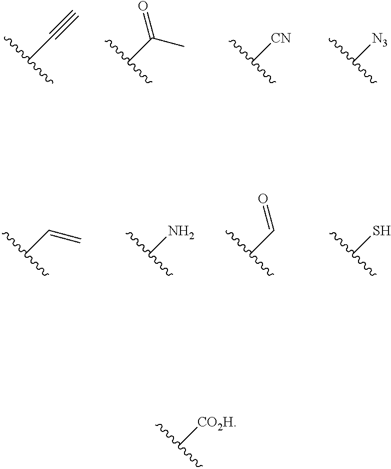

The term “pharmaceutically acceptable salt” includes a salt of a compound that does not cause significant irritation to an organism to which it is administered and does not abrogate or substantially reduce the biological activity and properties of the compound. In some embodiments, the salt of the compound may enhance the biological activity and properties of the compound. In other embodiments, the salt may additionally enhance the structural integrity or chemical stability of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, or phosphoric acid. Pharmaceutical salts can also be obtained by reacting a compound with an organic acid such as aliphatic or aromatic carboxylic, phosphonic, phosphoric, sulfinic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine. In some embodiments, the polypeptide is an acetate salt.

In some embodiments, the peptide, e.g. a constrained peptide, is synthesized using solid phase peptide synthesis (i.e., the peptide is synthesized on, and then cleaved from, a resin). In some embodiments, the peptide is constrained while attached to a resin. In some embodiments, the peptide is constrained after cleavage from a resin.

As used herein, the term “resin” refers to a resin useful for solid phase peptide synthesis. Solid phase peptide synthesis is a well-known synthetic technique; see generally, Atherton, E., Sheppard, R. C. Solid Phase Peptide Synthesis: A Practical Approach, IRL Press, Oxford, England, 1989, and Stewart J. M., Young, J. D. Solid Phase Peptide Synthesis, 2nd edition, Pierce Chemical Company, Rockford, 1984. Exemplary resins which may be suitable for synthesizing constrained peptides include, but are not limited to:
(1) alkenyl resins (e.g., REM resin, vinyl sulfone polymer-bound resin, vinyl-polystyrene resin);
(2) amine functionalized resins (e.g., amidine resin, N-(4-Benzyloxybenzyl)hydroxylamine polymer bound, (aminomethyl)polystyrene, polymer bound (R)-(+)-a-methylbenzylamine, 2-Chlorotrityl Knorr resin, 2-N-Fmoc-Amino-dibenzocyclohepta-1,4-diene, polymer-bound resin, 4-[4-(1-Fmoc-aminoethyl)-2-methoxy-5-nitrophenoxy]butyramidomethyl-polystyrene resin, 4-Benzyloxybenzylamine, polymer-bound, 4-Carboxybenzenesulfonamide, polymer-bound, Bis(tert-butoxycarbonyl)thiopseudourea, polymer-bound, Dimethylaminomethyl-polystyrene, Fmoc-3-amino-3-(2-nitrophenyl)propionic acid, polymer-bound, N-Methyl aminomethylated polystyrene, PAL resin, Sieber amide resin, tert-Butyl N-(2-mercaptoethyl)carbamate, polymer-bound, Triphenylchloromethane-4-carboxamide polymer bound);
(3) benzhydrylamine (BHA) resins (e.g., 2-Chlorobenzhydryl chloride, polymer-bound, HMPB-benzhydrylamine polymer bound, 4-Methylbenzhydrol, polymer-bound, Benzhydryl chloride, polymer-bound, Benzhydrylamine polymer-bound);
(4) Br-functionalized resins (e.g., 4-(Benzyloxy)benzyl bromide polymer bound, 4-Bromopolystyrene, Brominated PPOA resin, Brominated Wang resin, Bromoacetal, polymer-bound, Bromopolystyrene, HypoGel® 200 Br, Polystyrene A-Br for peptidesynthesis, Selenium bromide, polymer-bound, TentaGel HL-Br, TentaGel MB-Br, TentaGel S-Br, TentaGel S-Br);
(5) Chloromethyl resins (e.g., 5-[4-(Chloromethyl)phenyl]pentyl]styrene, polymer-bound, 4-(Benzyloxy)benzyl chloride polymer bound, 4-Methoxybenzhydryl chloride, polymer-bound);
(6) CHO-functionalized resins (e.g., (4-Formyl-3-methoxyphenoxymethyl)polystyrene, (4-Formyl-3-methoxyphenoxymethyl)polystyrene, 3-Benzyloxybenzaldehyde, polymer-bound, 4-Benzyloxy-2,6-dimethoxybenzaldehyde, polymer-bound, Formylpolystyrene, HypoGel® 200 CHO, Indole resin, Polystyrene A-CH(OEt)₂, TentaGel HL-CH(OEt)₂);
(7) Cl functionalized resins (e.g., Benzoyl chloride polymer bound, (chloromethyl)polystyrene, Merrifield's resin);
(8) CO₂H functionalized resins (e.g., Carboxyethylpolystryrene, HypoGel® 200 COOH, Polystyrene AM-COOH, TentaGel HL-COOH, TentaGel MB—COOH, TentaGel S COOH);
(9) Hypo-Gel resins (e.g., HypoGel® 200 FMP, HypoGel® 200 PHB, HypoGel® 200 Trt-OH, HypoGel® 200 HMB);
(10) I-functionalized resins (e.g., 4-Iodophenol, polymer-bound, Iodopolystyrene); Janda-Jels™ (JandaJel^{ä}-Rink amide, JandaJel-NH₂, JandaJel-Cl, JandaJel-4-Mercaptophenol, JandaJel-OH, JandaJel-1-(3-Dimethylaminopropyl)-3-ethylcarbodiimide, JandaJel-1,3,4,6,7,8-hexahydro-2H-pyrimido-[1,2-a]pyrimidine, JandaJel-morpholine, JandaJel-polypyridine, JandaJel-Triphenylphosphine, JandaJel-Wang);
(11) MBHA resins (3[4′-(Hydroxymethyl)phenoxy]propionic acid-4-methylbenzhydrylamine resin, 4-(Hydroxymethyl)phenoxyacetic acid polymer-bound to MBHA resin, HMBA-4-methylbenzhydrylamine polymer bound, 4-Methylbenzhydrylamine hydrochloride polymer bound Capacity (amine));
(12) NH₂ functionalized resins ((Aminomethyl)polystyrene, (Aminomethyl)polystyrene, HypoGel® 200 NH2, Polystyrene AM-NH₂, Polystyrene Microspheres 2-aminoethylated, Polystyrol Microspheres 2-bromoethylated, Polystyrol Microspheres 2-hydroxyethylated, TentaGel HL-NH₂, Tentagel M Br, Tentagel M NH₂, Tentagel M OH, TentaGel MB-NH₂, TentaGel S-NH₂, TentaGel S-NH₂);
(13) OH-functionalized resins (e.g., 4-hydroxymethylbenzoic acid, polymer-bound, Hydroxymethyl Resins, OH-functionalized Wang Resins);
(14) oxime resins (e.g., 4-Chlorobenzophenone oxime polymer bound, Benzophenone oxime polymer bound, 4-Methoxybenzophenone oxime polymer bound);
(15) PEG resins (e.g., ethylene glycol polymer bound);
(16) Boc-/Blz peptide synthesis resins (e.g., Boc-Lys(Boc)-Lys[Boc-Lys(Boc)]-Cys(Acm)-b-Ala-O-PAM resin, Boc-Lys(Fmoc)-Lys[Boc-Lys(Fmoc)]-b-Ala-O-Pam resin, Boc-Lys(Boc)-Lys[Boc-Lys(Boc)]-Lys{Boc-Lys(Boc)-Lys[Boc-Lys(Boc)]}-b-Ala-O-PAM resin, Boc-Lys(Fmoc)-Lys[Boc-Lys(Fmoc)]-Lys{Boc-Lys(Fmoc)-Lys[Boc-Lys(Fmoc)]}-b-Ala-O-PAM resin, Boc-Lys(Boc)-Lys[Boc-Lys(Boc)]-Lys{Boc-Lys(Boc)-Lys[Boc-Lys(Boc)]}-Cys(Acm)-b-Ala-O-PAM resin, Preloaded PAM resins);
(17) Fmoc-/t-Bu peptide synthesis resins (e.g., Fmoc-Lys(Fmoc)-Lys[Fmoc-Lys(Fmoc)]-b-Ala-O-Wang resin, Fmoc-Lys(Fmoc)-Lys[Fmoc-Lys(Fmoc)]-Lys{Fmoc-Lys(Fmoc)-Lys[Fmoc-Lys(Fmoc)]}-b-Ala-O-Wang resin, Preloaded TentaGel® S Trityl Resins, Preloaded TentaGel® Resins, Preloaded Trityl Resins, Preloaded Wang Resins, Trityl Resins Preloaded with Amino Alcohols);
(18) thiol-functionalized resins (e.g., HypoGel® 200 S-Trt, Polystyrene AM-S-Trityl, TentaGel HL-S-Trityl, TentaGel MB-S-Trityl, TentaGel S-S-Trityl); and
(19) Wang resins (e.g., Fmoc-Ala-Wang resin, Fmoc-Arg(Pbf)-Wang resin, Fmoc-Arg(Pmc)-Wang resin, Fmoc-Asn(Trt)-Wang resin, Fmoc-Asp(OtBu)-Wang resin, Fmoc-Cys(Acm)-Wang resin, Fmoc-Cys(StBu)-Wang resin, Fmoc-Cys(Trt) Wang resin, Fmoc-Gln(Trt)-Wang resin, Fmoc-Glu(OtBu)-Wang resin, Fmoc-Gly-Wang resin, Fmoc-His(Trt)-Wang resin, Fmoc-Ile-Wang resin, Fmoc-Leu-Wang resin, Fmoc-Lys(Boc)-Wang resin, Fmoc-Met-Wang resin, Fmoc-D-Met-Wang resin, Fmoc-Phe-Wang resin, Fmoc-Pro-Wang resin, Fmoc-Ser(tBu)-Wang resin, Fmoc-Ser(Trt)-Wang resin, Fmoc-Thr(tBu)-Wang resin, Fmoc-Trp(Boc) Wang resin, Fmoc-Trp-Wang resin, Fmoc-Tyr(tBu)-Wang resin, Fmoc-Val-Wang resin).

A “suitable amino-protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl) 6 chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido) benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl) benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyl disilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl) methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthio phosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxy benzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxy naphthylmethypbenzenesulfonamide (DNMBS), benzylsulfonamide, trifluoromethyl sulfonamide, and phenacylsulfonamide.

A “suitable carboxylic acid protecting group” or “protected carboxylic acid,” as used herein, are well known in the art and include those described in detail in Greene (1999). Examples of suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

A “suitable hydroxyl protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetra hydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl) phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxy methyl)benzoate, 2,6-dichloro-4-methylphenoxyacetate, 2,6-dichloro-4-(1,1,3,3-tetra methylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethyl phosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-climethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

A “suitable thiol protecting group,” as used herein, are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected thiol groups further include, but are not limited to, thioesters, carbonates, sulfonates allyl thioethers, thioethers, silyl thioethers, alkyl thioethers, arylalkyl thioethers, and alkyloxyalkyl thioethers. Examples of suitable ester groups include formates, acetates, proprionates, pentanoates, crotonates, and benzoates. Specific examples of suitable ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl groups include trimethylsilyl, triethylsilyl, t-butyldimethylsilyl, t-butyldiphenylsilyl, triisopropylsilyl ether, and other trialkylsilyl ethers. Examples of suitable alkyl groups include methyl, benzyl, p-methoxybenzyl, 3,4-dimethoxybenzyl, trityl, t-butyl, and allyl ether, or derivatives thereof. Examples of suitable arylalkyl groups include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers. Conditions for adding and removing the aforementioned protecting groups, alone and in combinations, are known in the art.

In some embodiments, the constrained salt of a peptide, or constrained peptide, or pharmaceutically acceptable salts thereof, is an alpha-helical peptide. In some embodiments, the constrained salt of a peptide, or constrained peptide, or pharmaceutically acceptable salts thereof, is a substantially alpha-helical peptide. As used herein, the phrase “substantially alpha-helical” refers to a polypeptide adopting, on average, backbone (p, yv) dihedral angles in a range from about (−90°, −15°) to about (−35°, −70°). Alternatively, the phrase “substantially alpha-helical” refers to a constrained polypeptide adopting dihedral angles such that the W dihedral angle of one residue and the cp dihedral angle of the next residue sums, on average, about −80° to about −125°. In some embodiments, the constrained salt of a peptide, or constrained peptide, or pharmaceutically acceptable salts thereof, adopts dihedral angles such that the W dihedral angle of one residue and the p dihedral angle of the next residue sums, on average, about −100° to about −110°. In some embodiments, the constrained salt of a peptide, or constrained peptide, or pharmaceutically acceptable salts thereof, adopts dihedral angles such that the W dihedral angle of one residue and the p dihedral angle of the next residue sums, on average, about −105°.

The phrase “substantially alpha-helical” may also refer to a polypeptide having at least 50%, 60%, 70%, 80%, 90%, or 95% of the amino acids provided in the polypeptide chain in an alpha-helical conformation, or with dihedral angles as specified herein. In some embodiments, the constrained salt of a peptide, or constrained peptide, or pharmaceutically acceptable salts thereof, is, or is at least, 25%, 30%, 35%, 40%, 45%, 50%, 55%, 60%, 65%, 70%, 80%, 85%, 90%, 95%, 96%, 97%, 98%, 99%, or any value in between, in an alpha-helical conformation. In some embodiments, the constrained salt of a peptide, constrained polypeptide, or pharmaceutically acceptable salts thereof, displays at least 5%, 10%, 15%, 20%, 25%, 30%, 35%, 40%, 45%, 50%, 55%, 60%, 65%, 70%, 80%, 85%, 90%, 95%, 100%, or any value in between, greater alpha-helicity than the equivalent unconstrained salt of the peptide or unconstrained polypeptide. Confirmation of alpha-helical secondary structure may be ascertained by known analytical techniques, such as x-ray crystallography, electron crystallography, fiber diffraction, fluorescence anisotropy, circular dichroism (CD), and nuclear magnetic resonance (NMR) spectroscopy.

In some embodiments, the constrained peptide comprises a polymer having at least 4 natural and/or non-natural amino acids, and comprising at least two crosslinking moieties.

In some embodiments, the crosslinking moieties may be crosslinked to form a carbon-carbon bond, a carbon-nitrogen bond, a carbon-oxygen bond, a carbon-sulfur bond, an amide bond, a disulfide bond, or a cycloaddition. In some embodiments, the crosslinking moieties comprise an alkene, an alkyne, an azide, a sulfhydryl, a carboxylic acid, an amine, an epoxide, an aziridine, a nitrile, or an imine. This crosslinking promotes helicity of the peptide.

A constrained peptide comprises at least two, at least four, or at least six crosslinking moieties in the helical turn. In some embodiments, a constrained peptide comprises 2, 4, 6, or 8 crosslinking moieties. In some embodiments, the crosslinking moieties are at the i and i+3 position in the helical turn of the peptide, the i and i+4 position of the helical turn of the peptide, the i and i+7 position of the helical turn of the peptide, or a combination of these positions, for example, the 2 and 6 positions (i and i+4) and the 12 and 16 positions (i and i+4) of the helical turn of a peptide, or, the 2 and 6 positions (i and i+4) and 10 and 17 positions (i and i+7) of the helical turn of a peptide.

Techniques for amide bond formation are well known in the art, and include, for example, those discussed in Valeur, et al., Chem. Soc. Rev., Vol. 38, No. 2, pp. 606-631 (2009), Montalbetti, et al., Tetrahedron, Vol. 61, No. 46, pp. 10827-10852 (2005), and Bode, et al., Nature, Vol. 480, pp. 471-479 (2011), the entire contents of each of which are hereby incorporated by reference in their entirety. Disulfide bond formation comprises oxidizing two proximate sulfhydryl groups (—SH). Appropriate oxidizing agents are known in the art, and include, for example, those discussed in Andreu, et al., Methods Mol. Biol., Vol. 35, pp. 91-169 (1994), the entire contents of which is hereby incorporated by reference in its entirety. Appropriate techniques for carbon-oxygen bond formation and carbon-nitrogen bond formation are known in the art, and include, for example, those discussed in Hein, et al., Pharm Res., Vol. 25, No. 10, pp. 2216-2230 (2008), the entire contents of which is hereby incorporated by reference in its entirety. Appropriate techniques for cycloaddition reactions are known in the art, and include, for example, those discussed in Hein, et al., Pharm Res., Vol. 25, No. 10, pp. 2216-2230 (2008) and Padwa and Pearson, “The Chemistry of Heterocyclic Compounds, Synthetic Applications of 1,3-Dipolar Cycloaddition Chemistry Toward Heterocycles and Natural Products,” (2003, Wiley and Sons), the entire contents of each of which are hereby incorporated by reference in their entirety. Appropriate techniques for carbon-carbon bond formation are known in the art, and include, for example, those discussed in Farina, et al., Org. Proc. Res. Dev., Vol. 13, p. 250 (2009), Grubbs, et al., J. Am. Chem. Soc., Vol. 127, p. 17160 (2005), Williams, et al., Chinia, Vol. 69, p. 142 (2015), and Sanford, et al., Angew. Chem. Int. Ed., Vol. 39, p. 3451 (2000), the entire contents of each of which are hereby incorporated by reference in their entirety. In some embodiments, the reagent for carbon-carbon bond formation is a ring-closing metathesis catalyst. In some embodiments, the ring-closing metathesis catalyst is selected from benzylidene-bis (tricyclohexylphosphino)-dichlororuthenium, [1,3-bis-(2,4,6-trimethylphenyl)-2-imidazolidinylidene]dichloro(phenylmethylene)(tricyclohexylphosphino) ruthenium, dichloro(o-isopropoxyphenylmethylene)(tricyclohexylphosphine)ruthenium(II), and [1,3-Bis-(2,4,6-trimethylphenyl)-2-imidazolidinylidene]dichloro(o-isopropoxyphenyl methylene)ruthenium.

### Advantages in Several Embodiments

As described above and herein, some embodiments of the application provide compositions comprising a cons trained salt of a peptide. Some embodiments of the application provide compositions comprising a constrained salt of a peptide (or combination of peptides). Some embodiments of the application provide compositions comprising a constrained peptide, or a pharmaceutically acceptable salt thereof.

In some embodiments, the compositions have reduced levels of stabilizers and other additives that may cause undesired side effects, and yet still provide the desired conformational and chemical stability. In some embodiments, the composition provides stability in the eye, nasal cavity, mouth, epithelium and other tissues for up to 1, 3, 6, 12, 24 and 48 hours or longer. In some embodiments, the composition is formulated such that some or all of the ingredients do not evaporate, become absorbed, drained or otherwise eliminated after application to the eye or other region, and instead remain stable and active for several hours (e.g., 1-3 hours, 3-6 hours, 6-12 hours, 12-24 hours, and ranges therein). In some embodiments, the composition comprises a constrained salt of a peptide, for example Lacripep™ or the other sequences identified herein, where the constrained salt of the peptide is applied to the eye, and the constrained salt of the peptide is integrated into the lipid layer of the tear covering the eye, or at the interface of the lipid and aqueous components of the tear, where the constrained salt of the peptide stabilizes the tear and remains in the tear for a period of at least 1-3 hours, at least 3-6 hours, or at least 12-24 hours, or more than 24 hours. In some embodiments, the composition comprises a constrained peptide, for example constrained Lacripep™ or the other constrained sequences identified herein, where the constrained peptide is applied to the eye, and the constrained peptide is integrated into the lipid layer of the tear covering the eye, or at the interface of the lipid and aqueous components of the tear, where the constrained peptide stabilizes the tear and remains in the tear for a period of at least 1-3 hours, at least 3-6 hours, or at least 12-24 hours, or more than 24 hours. This feature, in several embodiments, is particularly advantageous because it allows an active ingredient (such as a peptide or constrained peptide) to remain stable and efficacious for prolonged periods of time. In some embodiments, reduced frequency of administration results in an overall reduced overall burden of ingredients to sensitive areas of the body (such as the eye). In some embodiments, the composition comprises a pharmaceutically acceptable salt of a constrained peptide (or combination of constrained peptides).

Although constrained salts of peptides, constrained peptides, or pharmaceutically acceptable salts thereof, are provided in several embodiments herein, other compounds may be used as the active ingredient in addition to a peptide.

Peptides are highly selective and efficacious and, at the same time, relatively safe and well tolerated. Constrained salts of peptides, constrained peptides, or pharmaceutically acceptable salts thereof, are particularly well suited for the compositions described herein because constrained salts of peptides, constrained peptides, or pharmaceutically acceptable salts thereof, may have increased chemical and physical stability, relative to unconstrained salts of the peptides or unconstrained peptides. For example, unconstrained salts of peptides or unconstrained peptides are more prone to hydrolysis, oxidation, and aggregation than their constrained salt or constrained equivalents. Polypeptide compositions are typically aqueous solutions containing the active peptide along with numerous stabilizers, preservatives, and other agents to maintain the efficacy of the peptide. The stabilizers, preservatives, and other agents may maintain the chemical and/or structural integrity of the polypeptide, thus preserving its efficacy. Certain additives, such as stabilizers and preservatives, may cause undesirable side-effects, including hypersensitivity reactions, itching, and stinging or burning. However, to maximize the shelf-life of the peptide and maintain efficacy, these additives are required in most peptide compositions in amounts that cause undesired results. Even in compositions with all these additives, peptide therapeutics must typically be refrigerated, making transportation difficult, and, even with refrigeration, still have a short shelf-life. Moreover, as the peptides degrade and/or aggregate over time (especially through warming and cooling when taken from cold storage to room temperature for use), the by-products may not only be inactive, they may be toxic and/or immunogenic. Formulators may attempt to increase potency of peptide compositions by increasing the amount of the active peptide in the composition. However, increased peptide concentration also increases the rate of peptide aggregation and inactivation.

Thus, several embodiments herein provide peptide compositions that provide therapeutic amounts of constrained salts of peptides (or combinations of peptides), constrained peptides (or combinations of constrained peptides), or pharmaceutically acceptable salts thereof, are stable at room temperature, and contain reduced (e.g., only trace amounts) of stabilizers and/or preservatives, or none at all.

In some embodiments, the constrained peptide is a constrained form of the amino acid sequence (a) Ac-KQFIENGSEFAQKLLKKFS-NH₂, or Ac-Lys-Gln-Phe-Ile-Glu-Asn-Gly-Ser-Glu-Phe-Ala-Gln-Lys-Leu-Leu-Lys-Lys-Phe-Ser-NH₂, where “Ac” represents an N-terminal acetyl group and the C-terminus is amidated (SEQ ID NO: 1); or, (b) Ac-KQFIENGSEFAQKLLKKFSLLKPWA-NH₂, or Ac-Lys-Gln-Phe-Ile-Glu-Asn-Gly-Ser-Glu-Phe-Ala-Gln-Lys-Leu-Leu-Lys-Lys-Phe-Ser-Leu-Leu-Lys-Pro-Trp-Ala-NH₂, where “Ac” represents an acetyl group and the C-terminus is amidated (SEQ ID NO: 2). In some embodiments, the C-terminal, N-terminal or both the C- and N-terminal of SEQ ID NO: 1 or 2 are not modified.

In some embodiments, the peptide is a constrained salt of the amino acid sequence (a) Ac-KQFIENGSEFAQKLLKKFS-NH₂, or Ac-Lys-Gln-Phe-Ile-Glu-Asn-Gly-Ser-Glu-Phe-Ala-Gln-Lys-Leu-Leu-Lys-Lys-Phe-Ser-NH₂, where “Ac” represents an N-terminal acetyl group and the C-terminus is amidated (SEQ ID NO: 1); or, (b) Ac-KQFIENGSEFAQKLLKKFSLLKPWA-NH₂, or Ac-Lys-Gln-Phe-Ile-Glu-Asn-Gly-Ser-Glu-Phe-Ala-Gln-Lys-Leu-Leu-Lys-Lys-Phe-Ser-Leu-Leu-Lys-Pro-Trp-Ala-NH₂, where “Ac” represents an acetyl group and the C-terminus is amidated (SEQ ID NO: 2). In some embodiments, the C-terminal, N-terminal or both the C- and N-terminal of SEQ ID NO: 1 or 2 are not modified.

In some embodiments, the peptide or constrained peptide is selected from the group consisting of the amino acid sequence:

(a)
(SEQ ID NO: 10)
Ac-XQFIXNGSEFAQKLLKKFS-NH₂
(b)
(SEQ ID NO: 11)
Ac-XQFIENGXEFAQKLLKKFS-NH₂
(c)
(SEQ ID NO: 12)
Ac-KXFIEXGSEFAQKLLKKFS-NH₂
(d)
(SEQ ID NO: 13)
Ac-KXFIENGSXFAQKLLKKFS-NH₂
(e)
(SEQ ID NO: 14)
Ac-KQXIENXSEFAQKLLKKFS-NH₂
(f)
(SEQ ID NO: 15)
Ac-KQXIENGSEXAQKLLKKFS-NH₂
(g)
(SEQ ID NO: 16)
Ac-KQFXENGXEFAQKLLKKFS-NH₂
(h)
(SEQ ID NO: 17)
Ac-KQFXENGSEFXQKLLKKFS-NH₂
(i)
(SEQ ID NO: 18)
Ac-KQFIXNGSXFAQKLLKKFS-NH₂
(j)
(SEQ ID NO: 19)
Ac-KQFIXNGSEFAXKLLKKFS-NH₂
(k)
(SEQ ID NO: 20)
Ac-KQFIEXGSEXAQKLLKKFS-NH₂
(l)
(SEQ ID NO: 21)
Ac-KQFIEXGSEFAQXLLKKFS-NH₂
(m)
(SEQ ID NO: 22)
Ac-KQFIENXSEFXQKLLKKFS-NH₂
(n)
(SEQ ID NO: 23)
Ac-KQFIENXSEFAQKXLKKFS-NH₂
(o)
(SEQ ID NO: 24)
Ac-KQFIENGXEFAXKLLKKFS-NH₂
(p)
(SEQ ID NO: 25)
Ac-KQFIENGXEFAQKLXKKFS-NH₂
(q)
(SEQ ID NO: 26)
Ac-KQFIENGSXFAQXLLKKFS-NH₂
(r)
(SEQ ID NO: 27)
Ac-KQFIENGSXFAQKLLXKFS-NH₂
(s)
(SEQ ID NO: 28)
Ac-KQFIENGSEXAQKXLKKFS-NH₂
(t)
(SEQ ID NO: 29)
Ac-KQFIENGSEXAQKLLKXFS-NH₂
(u)
(SEQ ID NO: 30)
Ac-KQFIENGSEFXQKLXKKFS-NH₂
(v)
(SEQ ID NO: 31)
Ac-KQFIENGSEFXQKLLKKXS-NH₂
(w)
(SEQ ID NO: 32)
Ac-KQFIENGSEFAXKLLXKFS-NH₂
(x)
(SEQ ID NO: 33)
Ac-KQFIENGSEFAXKLLKKFX-NH₂
(y)
(SEQ ID NO: 34)
Ac-KQFIENGSEFAQXLLKXFS-NH₂
(z)
(SEQ ID NO: 35)
Ac-KQFIENGSEFAQKXLKKXS-NH₂
(aa)
(SEQ ID NO: 36)
Ac-KQFIENGSEFAQKLXKKFX-NH₂
(bb)
(SEQ ID NO: 37)
Ac-XQFXENGSEFAQKLLKKFS-NH₂
(cc)
(SEQ ID NO: 38)
Ac-KXFIXNGSEFAQKLLKKFS-NH₂
(dd)
(SEQ ID NO: 39)
Ac-KQXIEXGSEFAQKLLKKFS-NH₂
(ee)
(SEQ ID NO: 40)
Ac-KQFXENXSEFAQKLLKKFS-NH₂
(ff)
(SEQ ID NO: 41)
Ac-KQFIXNGXEFAQKLLKKFS-NH₂
(gg)
(SEQ ID NO: 42)
Ac-KQFIEXGSXFAQKLLKKFS-NH₂
(hh)
(SEQ ID NO: 43)
Ac-KQFIENXSEXAQKLLKKFS-NH₂
(ii)
(SEQ ID NO: 44)
Ac-KQFIENGXEFXQKLLKKFS-NH₂
(jj)
(SEQ ID NO: 45)
Ac-KQFIENGSXFAXKLLKKFS-NH₂
(kk)
(SEQ ID NO: 46)
Ac-KQFIENGSEXAQXLLKKFS-NH₂
(ll)
(SEQ ID NO: 47)
Ac-KQFIENGSEFXQKXLKKFS-NH₂
(mm)
(SEQ ID NO: 48)
Ac-KQFIENGSEFAXKLXKKFS-NH₂
(nn)
(SEQ ID NO: 49)
Ac-KQFIENGSEFAQXLLXKFS-NH₂
(oo)
(SEQ ID NO: 50)
Ac-KQFIENGSEFAQKXLKXFS-NH₂
(pp)
(SEQ ID NO: 51)
Ac-KQFIENGSEFAQKLXKKXS-NH₂
(qq)
(SEQ ID NO: 52)
Ac-KQFIENGSEFAQKLLXKFX-NH₂

where “Ac” represents an acetyl group at the N-terminus and the C-terminus is amidated, and “X” represents an amino acid comprising a crosslinking moiety. In some embodiments, X comprises a compound of Formula (I). The constrained form of the peptide has a covalent bond between the two X amino acids which promotes a helical conformation of the peptide, preferably an alpha helix. In some embodiments, the C-terminal, N-terminal or both the C- and N-terminal of the sequences (a)-(qq) listed above are not modified. In some embodiments, the peptide is a salt having of one of the sequences above, or a fragment thereof, optionally with the N-terminus acetylated and/or the C-terminus amidated.

In some embodiments, the peptide, or pharmaceutically acceptable salt thereof, is a constrained peptide having the amino acid sequence of one of the following, or a fragment thereof, optionally with the N-terminus acetylated and/or the C-terminus amidated, wherein at least two amino acids in the i and i+3, i and i+4, i and i+7 of the helical turn, or a combination of these positions, are replaced or modified, with compounds comprising a crosslinking moiety. The constrained form of the peptide has a covalent bond between the two amino acids comprising crosslinking moieties, where the bond promotes a helical conformation of the peptide, preferably an alpha helix. In some embodiments, the peptide is a constrained salt having the amino acid sequence of one of the following, or a fragment thereof, optionally with the N-terminus acetylated and/or the C-terminus amidated.

```
<210> SEQ ID NO 3
<211> LENGTH: 138
<212> TYPE: PRT
<213> ORGANISM: Homo sapiens
<400> SEQUENCE: 3
Met Lys Phe Thr Thr Leu Leu Phe Leu Ala Ala Val Ala Gly Ala Leu
1 5 10 15
Val Tyr Ala Glu Asp Ala Ser Ser Asp Ser Thr Gly Ala Asp Pro Ala
                        20 25 30
Gln Glu Ala Gly Thr Ser Lys Pro Asn Glu Glu Ile Ser Gly Pro Ala
                35 40 45
Glu Pro Ala Ser Pro Pro Glu Thr Thr Thr Thr Ala Gln Glu Thr Ser
        50 55 60
Ala Ala Ala Val Gln Gly Thr Ala Lys Val Thr Ser Ser Arg Gln Glu
65 70 75 80
Leu Asn Pro Leu Lys Ser Ile Val Glu Lys Ser Ile Leu Leu Thr Glu
                                85 90 95
Gln Ala Leu Ala Lys Ala Gly Lys Gly Met His Gly Gly Val Pro Gly
                        100 105 110
Gly Lys Gln Phe Ile Glu Asn Gly Ser Glu Phe Ala Gln Lys Leu Leu
                115 120 125
Lys Lys Phe Ser Leu Leu Lys Pro Trp Ala
        130 135
<210> SEQ ID NO 4
<211> LENGTH: 119
<212>
TYPE: PRT
<213> ORGANISM: Homo sapiens
<400> SEQUENCE: 4
Glu Asp Ala Ser Ser Asp Ser Thr Gly Ala Asp Pro Ala Gln Glu Ala
1 5 10 15
Gly Thr Ser Lys Pro Asn Glu Glu Ile Ser Gly Pro Ala Glu Pro Ala
                        20 25 30
Ser Pro Pro Glu Thr Thr Thr Thr Ala Gln Glu Thr Ser Ala Ala Ala
                35 40 45
Val Gln Gly Thr Ala Lys Val Thr Ser Ser Arg Gln Glu Leu Asn Pro
        50 55 60
Leu Lys Ser Ile Val Glu Lys Ser Ile Leu Leu Thr Glu Gln Ala Leu
65 70 75 80
Ala Lys Ala Gly Lys Gly Met His Gly Gly Val Pro Gly Gly Lys Gln
                                85 90 95
Phe Ile Glu Asn Gly Ser Glu Phe Ala Gln Lys Leu Leu Lys Lys Phe
                        100 105 110
Ser Leu Leu Lys Pro Trp Ala
                115
<210> SEQ ID NO 5
<211> LENGTH: 114
<212> TYPE: PRT
<213> ORGANISM: homo sapiens
<400> SEQUENCE: 5
Asp Ser Thr Gly Ala Asp Pro Ala Gln Glu Ala Gly Thr Ser Lys Pro
1 5 10 15
Asn Glu Glu Ile Ser Gly Pro Ala Glu Pro Ala Ser Pro Pro Glu Thr
                        20 25 30
Thr Thr Thr Ala Gln Glu Thr Ser Ala Ala Ala Val Gln Gly Thr Ala
                35 40 45
Lys Val Thr Ser Ser Arg Gln Glu Leu Asn Pro Leu Lys Ser Ile Val
        50 55 60
Glu Lys Ser Ile Leu Leu Thr Glu Gln Ala Leu Ala Lys Ala Gly Lys
65 70 75 80
Gly Met His Gly Gly Val Pro Gly Gly Lys Gln Phe Ile Glu Asn Gly
                                85 90 95
Ser Glu Phe Ala Gln Lys Leu Leu Lys Lys Phe Ser Leu Leu Lys Pro
                        100 105 110
Trp Ala
<210> SEQ ID NO 6
<211> LENGTH: 114
<212> TYPE: PRT
<213> ORGANISM: Homo sapiens
<400> SEQUENCE: 6
Glu Asp Ala Ser Ser Asp Ser Thr Gly Ala Asp Pro Ala Gln Glu Ala
1 5 10 15
Gly Thr Ser Lys Pro Asn Glu Glu Ile Ser Gly Pro Ala Glu Pro Ala
                        20 25 30
Ser Pro Pro Glu Thr Thr Thr Thr Ala Gln Glu Thr Ser Ala Ala Ala
                35 40 45
Val Gln Gly Thr Ala Lys Val Thr Ser Ser Arg Gln Glu Leu Asn Pro
        50 55 60
Leu Lys Ser Ile Val Glu Lys Ser Ile Leu Leu Thr Glu Gln Ala Leu
65 70 75 80
Ala Lys Ala Gly Lys Gly Met His Gly Gly Val Pro Gly Gly Lys Gln
                                85 90 95
Phe Ile Glu Asn Gly Ser Glu Phe Ala Gln Lys Leu Leu Lys Lys Phe
                        100 105 110
Ser Leu
<210> SEQ ID NO 7
<211> LENGTH: 109
<212> TYPE: PRT
<213> ORGANISM: homo sapiens
<400> SEQUENCE: 7
Glu Asp Ala Ser Ser Asp Ser Thr Gly Ala Asp Pro Ala Gln Glu Ala
1 5 10 15
Gly Thr Ser Lys Pro Asn Glu Glu Ile Ser Gly Pro Ala Glu Pro Ala
                        20 25 30
Ser Pro Pro Glu Thr Thr Thr Thr Ala Gln Glu Thr Ser Ala Ala Ala
                35 40 45
Val Gln Gly Thr Ala Lys Val Thr Ser Ser Arg Gln Glu Leu Asn Pro
        50 55 60
Leu Lys Ser Ile Val Glu Lys Ser Ile Leu Leu Thr Glu Gln Ala Leu
65 70 75 80
Ala Lys Ala Gly Lys Gly Met His Gly Gly Val Pro Gly Gly Lys Gln
                                85 90 95
Phe Ile Glu Asn Gly Ser Glu Phe Ala Gln Lys Leu Leu
                        100 105
<210> SEQ ID NO 8
<211> LENGTH: 104
<212> TYPE: PRT
<213> ORGANISM: homo sapiens
<400> SEQUENCE: 8
Glu Asp Ala Ser Ser Asp Ser Thr Gly Ala Asp Pro Ala Gln Glu Ala
1 5 10 15
Gly Thr Ser Lys Pro Asn Glu Glu Ile Ser Gly Pro Ala Glu Pro Ala
                        20 25 30
Ser Pro Pro Glu Thr Thr Thr Thr Ala Gln Glu Thr Ser Ala Ala Ala
                35 40 45
Val Gln Gly Thr Ala Lys Val Thr Ser Ser Arg Gln Glu Leu Asn Pro
        50 55 60
Leu Lys Ser Ile Val Glu Lys Ser Ile Leu Leu Thr Glu Gln Ala Leu
65 70 75 80
Ala Lys Ala Gly Lys Gly Met His Gly Gly Val Pro Gly Gly Lys Gln
                                85 90 95
Phe Ile Glu Asn Gly Ser Glu Phe
                        100
<210> SEQ ID NO 9
<211> LENGTH: 14
<212> TYPE: PRT
<213> ORGANISM: homo sapiens
<400> SEQUENCE: 9
Asn Gly Ser Glu Phe Ala Gln Lys Leu Leu Lys Lys Phe Ser
1 5 10
```

In some embodiments, the constrained peptide is a constrained form of the peptide having the amino acid sequence Ac-KQFIENGSEFAQKLLKKFS-NH₂ or Ac-Lys-Gln-Phe-Ile-Glu-Asn-Gly-Ser-Glu-Phe-Ala-Gln-Lys-Leu-Leu-Lys-Lys-Phe-Ser-NH₂, where “Ac” represents an N-terminal acetyl group and the C-terminus is amidated (SEQ ID NO: 1). In some embodiments, the peptide is constrained Lacripep™, or a pharmaceutically acceptable salt thereof. In some embodiments, the constrained peptide is any one or more of SEQ IDs 1-9, wherein at least two amino acids in the i and i+3, i and i+4, i and i+7 of the helical turn, or a combination of these positions, are replaced or modified with compounds comprising a crosslinking moiety, and a covalent bond is formed between the moieties which promotes a helical conformation, preferably an alpha helix. In some embodiments, the C-terminal, N-terminal or both the C- and N-terminal of the peptide are not modified.

In some embodiments, the constrained salt of the peptide is represented by the amino acid sequence Ac-KQFIENGSEFAQKLLKKFS-NH₂ or Ac-Lys-Gln-Phe-Ile-Glu-Asn-Gly-Ser-Glu-Phe-Ala-Gln-Lys-Leu-Leu-Lys-Lys-Phe-Ser-NH₂, where “Ac” represents an N-terminal acetyl group and the C-terminus is amidated (SEQ ID NO: 1). In some embodiments, the peptide is a constrained salt of Lacripep™. In some embodiments, the peptide is a constrained salt of any one or more of SEQ IDs 1-9. In some embodiments, the C-terminal, N-terminal or both the C- and N-terminal of the peptide are not modified.

### Buffers and pH

Buffers stabilize the pH of a solution, i.e., resist changes in pH when acidic or alkaline materials are added to the solution. Suitable buffers for use in the present composition include, but are not limited to, glycine hydrochloride, sodium acetate, phosphate buffered saline (PBS) (including mono- and dihydrogen phosphate salts), citrate buffer (citric acid and sodium citrate), phosphate-citrate buffer, tris(hydroxymethyl)aminomethane (Tris), carbonate buffers (sodium carbonate and sodium bicarbonate), borate buffers, and combinations thereof.

In some embodiments, the buffer comprises one or more of sodium acetate, phosphate buffered saline (PBS), citrate buffer (citric acid and sodium citrate), and phosphate-citrate buffer. In some embodiments, the buffer is selected from the group consisting of sodium acetate, phosphate buffered saline (PBS), citrate buffer (citric acid and sodium citrate), and phosphate-citrate buffer.

In some embodiments, the amount of buffer is limited to less than 0.1, 0.2, 0.3, or, 0.4%, or within a range defined by any two of the preceding values.

In an embodiment, the buffer is a citrate buffer (citric acid and sodium citrate). In an embodiment, the only buffer is a citrate buffer, and no other buffering agent is present in the composition.

In some embodiments the pH of the composition is between 6 to 7.4; 6.1 to 7.3; 6.2 to 7.2; 6.3 to 7.1; 6.4 to 7.0; 6.5 to 6.9; 6.6 to 6.8; or any pH in between. In some embodiments the pH of the composition is, or is about, 6; 6.1; 6.2; 6.3; 6.4; 6.5; 6.6; 6.7; 6.8; 6.9; 7; 7.1; 7.2; 7.3; 7.4, or a range defined by any two of the preceding values. In an embodiment, the pH of the composition is, or is about, 6.2, 6.3, 6.4, 6.5, 6.6, 6.7, or 6.8.

The pH of the composition can be adjusted as necessary by the addition of solutions of an acid or a base. Any acid or base whose conjugate is ophthalmically acceptable may be used. Acids include for example hydrochloric acid, bases include for example sodium and potassium hydroxides.

### Chelating Agents

In some embodiments, the composition further comprises one or more chelating agents. In some embodiments, the chelating agents are selected from the group consisting of ethylenediaminetetraacetic acid, edetate disodium (EDTA), ethylenediamine, amino acids such as glutamic acid and histidine, organic diacids such as oxalic acid, malonic acid, succinic acid, and the like, 3-dimercaptopropanesulfonic acid (DMPS), alpha lipoic acid (ALA), 2,3-dimercaptopropanesulfonic acid (DMPS), thiamine tetrahydrofurfuryl disulfide (TTFD), penicillamine, dimercaptosuccinic acid (DMSA), combinations thereof, and pharmaceutically acceptable salts of the foregoing.

In some embodiments, the chelating agent, as a non-limiting example EDTA, or a pharmaceutically acceptable salt thereof, is present at between 0.0001% and 0.1%; between 0.0005% and 0.05%; 0.0006% and 0.04%; 0.0007% and 0.003%; 0.0008% and 0.002%; 0.0009% and 0.001%; or any value contained there between. In some embodiments, the chelating agent is present at an amount that is, or is less than, 0.1%; 0.09%; 0.08%; 0.07%; 0.06%; 0.05%; 0.04%; 0.03%; 0.02%; 0.01%; 0.009%; 0.008%; 0.007%; 0.006%; 0.005%; 0.004%; 0.003%; 0.002%; 0.001%; 0.0009%; 0.0008%; 0.0007%; 0.0006%; 0.0005%; 0.0004%; 0.0003%; 0.0002%; or 0.0001%, or is within a range defined by any two of the preceding values.

In some embodiments, the chelating agent, such as EDTA or others, or a pharmaceutically acceptable salt thereof, is present at less than about 0.05% or less than about 0.005% (e.g., at about 0.001%).

### Stabilizing Agents

Buffers and chelators can stabilize peptide ingredients of compositions by maintaining pH and reducing metal ion mediated degradation of the peptides. In some embodiments, the composition further comprises one or more peptide stabilizing agents in addition to a buffer and/or a chelating agent. In some embodiments, the one or more stabilizing agents in addition to a buffer and/or chelating agent are selected from the group consisting of disaccharides, polysaccharides (e.g., hyaluronic acid), polyols, sugar alcohols, amino acids, proteins (e.g., serum albumin), and combinations thereof. In some embodiments, non-limiting examples of stabilizers include trehalose, sucrose, mannitol, sorbitol, polysorbate 20, polysorbate 80, histidine, glycine, and arginine, and combinations thereof. In an embodiment the composition does not include a stabilizer in addition to a buffering agent and/or a chelator.

### Polypeptide Degradation

Polypeptides are prone to physical and chemical degradation, both during storage and after administration, For example, aggregation, shearing, oxidation, deamidation, and hydrolysis. Liquid peptide compositions in particular have a high risk for physical and chemical instability during manufacturing and storage. Reducing polypeptide degradation is particularly important for dilute peptide formulations, which initially contain very small amounts of a particular peptide. Loss of even miniscule amounts of the initial small amount can significantly impact the efficacy of the composition.

In some embodiments, composition stability is determined by high-performance liquid chromatography (HPLC). In some embodiments, composition stability is determined by high-performance liquid chromatography-mass spectrometry (HPLC-MS). Some embodiments provide constrained peptides (or combinations of constrained peptides) or pharmaceutically acceptable salts thereof, that are more resistant to degradation than the equivalent unconstrained peptide (or combination of peptides). Some embodiments provide constrained salts of peptides (or combinations of constrained salts of peptides) or pharmaceutically acceptable salts thereof, that are more resistant to degradation than the equivalent unconstrained salts of the peptide (or combination of peptides).

In some embodiments, composition stability is determined after a sealed container of the composition has been in the dark, or exposed to light, at room temperature for days, weeks or months (e.g., 1-24 days or months, e.g., 1, 2, 3, 4, 5, 6, 7, 8, 9, 10, 11, 12, 13, 14, 15, 16, 17, 18, 19, 20, 21, 22, 23, 24 days or months). In some embodiments, composition stability is determined after a sealed container of the composition has been in the dark, or exposed to light, at 2 to 8° C., for example 5° C., or any value in between, for days, weeks or months, (e.g., 1-24 days or months, e.g., 1, 2, 3, 4, 5, 6, 7, 8, 9, 10, 11, 12, 13, 14, 15, 16, 17, 18, 19, 20, 21, 22, 23, 24 days or months) In some embodiments, composition stability is determined after a sealed container of the composition has been in the dark, or exposed to light, at −10 to −30° C., for example −25° C., or any value in between, for days, weeks or months, (e.g., 1-24 days or months, e.g., 1, 2, 3, 4, 5, 6, 7, 8, 9, 10, 11, 12, 13, 14, 15, 16, 17, 18, 19, 20, 21, 22, 23, 24 days or months) In some embodiments, composition stability is determined after a sealed container of the composition has been in the dark, or exposed to light, at 20 to 30° C., for example 25° C., or any value in between, for days, weeks or months, (e.g., 1-24 days or months, e.g., 1, 2, 3, 4, 5, 6, 7, 8, 9, 10, 11, 12, 13, 14, 15, 16, 17, 18, 19, 20, 21, 22, 23, 24 days or months) In some embodiments, composition stability is determined after a sealed container of the composition has been in the dark, or exposed to light, and moved from 2 to 8° C. (storage), or any value in between, to room temperature for 5 minutes, either one, two, or three times per day, for 1-60 days.

In some embodiments, the composition provides at least 99%; 98%; 97%; 96%; 95%; 94%; 93%; 92%; 91%; 90%; 89%; 88%; 87%; 86%; 85%; 84%; 83%; 82%; 81%; 80%; 79%; 78%; 77%; 76%; 75%; 74%; 73%; 72%; 71%; 70%; or any value in between, of the original amount or activity of the constrained polypeptide, or a pharmaceutically acceptable salt thereof, in an intact, non-degraded or non-aggregated form of the peptide, following exposure to one or more of the conditions described above and herein. In a one embodiment, the amount or activity of the intact constrained polypeptide, or a pharmaceutically acceptable salt thereof, is at least 80%, 85%, 90% or 95% of the original amount. In some embodiments, the amount or activity of intact polypeptide, or a pharmaceutically acceptable salt thereof, is at least 97% of the original amount.

In some embodiments, the composition provides at least 99%; 98%; 97%; 96%; 95%; 94%; 93%; 92%; 91%; 90%; 89%; 88%; 87%; 86%; 85%; 84%; 83%; 82%; 81%; 80%; 79%; 78%; 77%; 76%; 75%; 74%; 73%; 72%; 71%; 70%; or any value in between, of the original amount or activity of the constrained salt of the polypeptide, or a pharmaceutically acceptable salt thereof, in an intact, non-degraded or non-aggregated form of the peptide, following exposure to one or more of the conditions described above and herein. In a one embodiment, the amount or activity of the intact constrained salt of the polypeptide, or a pharmaceutically acceptable salt thereof, is at least 80%, 85%, 90% or 95% of the original amount. In some embodiments, the amount or activity of intact polypeptide, or a pharmaceutically acceptable salt thereof, is at least 97% of the original amount.

In some embodiments, the composition provides at least 1%; 2%; 3%; 4%; 5%; 6%; 7%; 8%; 9%; 10%; 11%; 12%; 13%; 14%; 15%; 16%; 17%; 18%; 19%; 20%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%, or any value in between, more activity than the original amount or activity of the unconstrained polypeptide, or a pharmaceutically acceptable salt thereof, in an intact, non-degraded or non-aggregated form, following exposure to one or more of the conditions described above and herein. In a one embodiment, the amount or activity of the constrained polypeptide, or a pharmaceutically acceptable salt thereof, is at least 5%, 10%, 15%, 20%, 25%, 30%, 35%, 40%, 45%, or 50% greater of the activity of the unconstrained polypeptide, or a pharmaceutically acceptable salt thereof.

In some embodiments, the composition provides at least 1%; 2%; 3%; 4%; 5%; 6%; 7%; 8%; 9%; 10%; 11%; 12%; 13%; 14%; 15%; 16%; 17%; 18%; 19%; 20%; 21%; 22%; 23%; 24%; 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%, or any value in between, more activity than the original amount or activity of the unconstrained salt of the polypeptide, or a pharmaceutically acceptable salt thereof, in an intact, non-degraded or non-aggregated form, following exposure to one or more of the conditions described above and herein. In a one embodiment, the amount or activity of the constrained salt of the polypeptide, or a pharmaceutically acceptable salt thereof, is at least 5%, 10%, 15%, 20%, 25%, 30%, 35%, 40%, 45%, or 50% greater of the activity of the unconstrained salt of the polypeptide, or a pharmaceutically acceptable salt thereof.

In some embodiments, the composition comprises not more than 30%; 29%; 28%; 27%; 26%; 25%; 24%; 23%; 22%; 21%; 20%; 19%; 18%; 17%; 16%; 15%; 14%; 13%; 12%; 11%; 10%; 9%; 8%; 7%; 6%; 5%; 4%; 3%; 2%; 1%; of the total amount of the constrained peptide in the composition is in the form of a particular aggregation product and/or a particular degradation product, or is within a range defined by any two of the preceding values, following exposure to one or more of the conditions described above and herein. In some embodiments, the composition comprises not more than about 15%, or not more than 20%, inactive constrained peptide.

In some embodiments, the composition comprises not more than 30%; 29%; 28%; 27%; 26%; 25%; 24%; 23%; 22%; 21%; 20%; 19%; 18%; 17%; 16%; 15%; 14%; 13%; 12%; 11%; 10%; 9%; 8%; 7%; 6%; 5%; 4%; 3%; 2%; 1%; of the total amount of the constrained form of a peptide in the composition is in the form of a particular aggregation product and/or a particular degradation product, or is within a range defined by any two of the preceding values, following exposure to one or more of the conditions described above and herein. In some embodiments, the composition comprises not more than about 15%, or not more than 20%, inactive constrained form of a peptide.

In some embodiments, the composition comprises not more than 30%; 29%; 28%; 27%; 26%; 25%; 24%; 23%; 22%; 21%; 20%; 19%; 18%; 17%; 16%; 15%; 14%; 13%; 12%; 11%; 10%; 9%; 8%; 7%; 6%; 5%; 4%; 3%; 2%; 1%; of the total amount of the constrained salt of a peptide in the composition is in the form of a particular aggregation product and/or a particular degradation product, or is within a range defined by any two of the preceding values, following exposure to one or more of the conditions described above and herein. In some embodiments, the composition comprises not more than about 15%, or not more than 20%, inactive constrained salt of a peptide.

In some embodiments, the composition comprises not more than 30%; 29%; 28%; 27%; 26%; 25%; 24%; 23%; 22%; 21%; 20%; 19%; 18%; 17%; 16%; 15%; 14%; 13%; 12%; 11%; 10%; 9%; 8%; 7%; 6%; 5%; 4%; 3%; 2%; 1%; of the total amount of constrained peptide in the composition is in the form of any degradation product and/or aggregation product, or is within a range defined by any two of the preceding values, following exposure to one or more of the conditions described above and herein.

In some embodiments, the composition comprises not more than 30%; 29%; 28%; 27%; 26%; 25%; 24%; 23%; 22%; 21%; 20%; 19%; 18%; 17%; 16%; 15%; 14%; 13%; 12%; 11%; 10%; 9%; 8%; 7%; 6%; 5%; 4%; 3%; 2%; 1%; of the total amount of the constrained salt of a peptide in the composition is in the form of any degradation product and/or aggregation product, or is within a range defined by any two of the preceding values, following exposure to one or more of the conditions described above and herein.

In some embodiments, the composition comprises very low levels of buffer, in combination with very low levels of a chelator. In some embodiments, the buffer is a citrate buffer and the chelator is EDTA.

Embodiments of compositions of a constrained salt of a peptide or a constrained peptide described herein provide advantages in manufacturing, transportation, storage, and use of the constrained salt of a peptide or constrained peptide compositions by decreasing peptide aggregation and degradation, thus maintaining the efficacy of the compositions and reducing buildup of undesired breakdown products in the composition.

In some embodiments, the constrained salt of a peptide composition reduces the rate of formation of breakdown and/or aggregation products. In some embodiments, the constrained peptide composition reduces the rate of formation of breakdown and/or aggregation products.

In some embodiments, the constrained peptide is constrained Lacripep™ or a pharmaceutically acceptable salt thereof. In some embodiments, the composition comprises less than about 5%, 4%, 3%, 2%, or about 1% total degradation products. In some embodiments, the stabilized composition comprises not more than 0.25%, 0.5%, 0.75%, 1.0%, 1.25%, 1.5%, 1.75%, or 2.0% of any single degradation product. In some embodiments, the stabilized composition comprises less than about 5%, 4%, 3%, 2%, or about 1% total degradation products and not more than 0.25%, 0.5%, 0.75%, 1.0%, 1.25%, 1.5%, 1.75%, or 2.0% of any single degradation product.

In some embodiments, the constrained salt of a peptide is a constrained salt of Lacripep™, or a pharmaceutically acceptable salt thereof. In some embodiments, the composition comprises less than about 5%, 4%, 3%, 2%, or about 1% total degradation products. In some embodiments, the stabilized composition comprises not more than 0.25%, 0.5%, 0.75%, 1.0%, 1.25%, 1.5%, 1.75%, or 2.0% of any single degradation product. In some embodiments, the stabilized composition comprises less than about 5%, 4%, 3%, 2%, or about 1% total degradation products and not more than 0.25%, 0.5%, 0.75%, 1.0%, 1.25%, 1.5%, 1.75%, or 2.0% of any single degradation product.

In some embodiments, the aggregation products include dimers, trimers, tetramers, or larger-order peptide aggregates.

### Preservatives

In some embodiments, the composition further comprises one or more preservatives to prevent the growth of microbes in the composition. In some embodiments, the composition further comprises one or more preservatives to maintain the sterility of the composition. In some embodiments, the composition further comprises one or more preservatives to prevent the growth of microbes and maintain the sterility of the composition. However, in many embodiments, the preservative is provided in reduced amounts. In some embodiments, the one or more preservatives are selected from the group consisting of benzalkonium chloride, cetylpyridinium chloride, chlorobutanol, benzododecinium bromide, methylparaben, propylparaben, phenylethyl alcohol, sodium perborate, edentate disodium, chlorobutanol, sorbic acid, benzethonium chloride, sodium acetate, polyquaternium-1, phenylmercuric nitrate, phenylmercury borate, sodium propionate, chlorhexidine, thimerosal, and combinations thereof. In some embodiments, the composition does not contain a preservative. In some embodiments, the composition does not contain detectable levels of a preservative. In some embodiments, the constrained polypeptide, or pharmaceutically acceptable salt thereof, can be self-preserving, i.e., no additional preservatives are necessary to maintain sterility of the composition. In some embodiments, the constrained salt of a polypeptide, or pharmaceutically acceptable salt thereof, can be self-preserving, i.e., no additional preservatives are necessary to maintain sterility of the composition.

In some embodiments, the preservative is present at between 0.0001% and 1%; between 0.01% and 0.9%; 0.05% and 0.8%; 0.1% and 0.7%; 0.2% and 0.3%; 0.4% or 0.5%, or any value contained there between. In some embodiments, the preservative is present in an amount that is, or is less than, 1%; 0.9%; 0.8%; 0.7%; 0.6%; 0.5%; 0.4%; 0.3%; 0.2%; 0.1%; 0.09%; 0.08%; 0.07%; 0.06%; 0.05%; 0.04%; 0.03%; 0.02%; 0.01%; 0.009%; 0.008%; 0.007%; 0.006%; 0.005%; 0.004%; 0.003%; 0.002%; or 0.001%, or is within a range defined by any two of the preceding values.

In some embodiments, the composition is sterile. In some embodiments, the composition is manufactured from sterile ingredients in an aseptic environment. In some embodiments, the composition is sterile and preservative free. In some embodiments, the composition is sterilized just prior to packaging. In some embodiments, the composition is sterilized by one or more of the following (1) addition of one or more quaternary ammonium chlorides to the composition; (2) exposing the composition to ionizing radiation; (3) filtering the composition; (4) exposing the composition to ionizing radiation after packaging; and any combination of the foregoing. In some embodiments, filtering comprises passing the composition through a filter (including but not limited to a 0.22 micron filter with a polyvinyldifluoride or other suitable membrane (e.g., polyethersulfone).

In some embodiments, the constrained salt of a peptide, constrained peptide, or pharmaceutically acceptable salt thereof, is provided in a bacteriostatic and/or bactericidal amount. In some embodiments, the amount of the constrained salt of a peptide, constrained peptide, or pharmaceutically acceptable salt thereof, provided in the composition is bacteriostatic and/or bactericidal when one, two or three drops of the composition are administered to the surface of the eye. In some embodiments, the constrained salt of a peptide, constrained peptide, or pharmaceutically acceptable salt thereof, is bacteriostatic and/or bactericidal for Gram-positive and/or Gram-negative bacteria, for example, when administered to the eye. In some embodiments the amount of the constrained salt of a peptide, constrained peptide, or pharmaceutically acceptable salt thereof, in the composition is sufficient to inhibit bacterial growth by at least 20%, 30%, 40%, 50%, 60%, 70%, 80% or 90% relative to a control composition not containing the peptide in a standard bacteriological assay. In some embodiments, the bacteria in the bacteriological assay are selected from P. aeruginoa, E. coli, S. epidermis, S. aureus, or combinations thereof. In some embodiments, the bacteriological assay is selected from a bacterial growth assay, SYTOX Green assay, a well diffusion assay, a broth or agar dilution assay, a time-kill test, antimicrobial gradient assay, a ATP-bioluminescence assay, or a propidium-iodide flow cytometry assay. In some embodiments, the constrained salt of a peptide, constrained peptide, or pharmaceutically acceptable salt thereof, provided in a bacteriostatic and/or bactericidal amount is a constrained salt or constrained form of Lacripep™.

In some embodiments, the bacteriological assay is a USP Section <51> assay or FDA-mandated assay. For example, the original product containers, containing the peptide solution, and inoculate each container with one of the prepared and standardized inoculums (e.g., P. aeruginoa, E. coli, S. epidermis, S. aureus, or combinations thereof) and mix. The volume of the suspension inoculums should be about 0.5% to 1.0% of the volume of the product, and the concentration of the test preparation immediately after inoculation is between 1×10⁵ and 1×10⁶ colony forming organisms (CFU) per mL of product (as measured by, for example, the plate count method, or another microbial enumeration test).

The inoculated containers are incubated at between 22.5±2.5° C. in a controlled environment and sampled at specified intervals, for example, 7, 14, and 28 days. Any change in appearance is recorded, and the CFU/mL are determined, at each sampling. The change in log₁₀ values of CFU/mL provides the change over time in terms of log reductions. The product provides not less than 1.0 log reduction from the initial calculated count at 7 days, not less than 3.0 log reduction from the initial count at 14 days, and no increase from the 14 day count at 28 days for bacteria, and no increase from the initial count of yeast and molds. In some embodiments, the constrained salt of a peptide provided in a bacteriostatic and/or bactericidal amount is a constrained salt of Lacripep™. In some embodiments, the constrained peptide provided in a bacteriostatic and/or bactericidal amount is constrained Lacripep™, or a pharmaceutically acceptable salt thereof.

### Surfactants

In some embodiments, the composition further comprises one or more surfactants. In some embodiments, the one or more surfactants are selected from detergents, wetting agents, emulsifiers, foaming agents, dispersants, and combinations thereof. In some embodiments, the surfactant comprises a constraining salt.

In some embodiments, the surfactant is an anionic surfactant. Anionic surfactants contain anionic functional groups at their head, such as sulfate, sulfonate, phosphate, and carboxylates. In some embodiments, the surfactant is a sulfate, sulfonate, or phosphate ester, e.g., a sulfate ester. In some embodiments, the surfactant is selected from the group comprising or consisting of ammonium lauryl sulfate and sodium lauryl sulfate, e.g., sodium lauryl sulfate (also called SDS, sodium dodecyl sulfate). In some embodiments, the surfactant is an alkyl-ether sulfate, such as selected from the group comprising or consisting of sodium laureth sulfate (also known as sodium lauryl ether sulfate), and sodium myreth sulfate. In some embodiments, the surfactant is a docusate, such as dioctyl sodium sulfosuccinate, perfluorooctanesulfonate (PFOS), perfluorobutanesulfonate, linear alkylbenzene sulfonates (LABs). In some embodiments, the surfactant is a carboxylate, such as alkyl carboxylates (soaps), for instance sodium stearate; sodium lauroyl sarcosinate and carboxylate-based fluorosurfactants such as perfluorononanoate, perfluorooctanoate (PFOA or PFO). In some embodiments, the constrained salt of a polypeptide contributes to the surfactant properties of the composition. In some embodiments, the constrained polypeptide, or pharmaceutically acceptable salt thereof, contributes to the surfactant properties of the composition.

In some embodiments, the surfactant is a cationic surfactant, of which the charge can be pH dependent, such as primary, secondary or tertiary amines, for instance octenidine dihydrochloride; or may comprise permanently charged quaternary ammonium cations, such as alkyltrimethylammonium salts, for instance cetyl trimethylammonium bromide (CTAB) or cetyl trimethylammonium chloride (CTAC); cetylpyridinium chloride (CPC); benzalkonium chloride (BAC); benzethonium chloride (BZT); 5-Bromo-5-nitro-1,3-dioxane; dimethyldioctadecylammonium chloride; or dioctadecyldimethylammonium bromide (DODAB). In some embodiments, the surfactant is a zwitterionic surfactant (i.e. having both cationic and anionic centers attached to the same molecule). The cationic part may be based on primary, secondary, or tertiary amines or quaternary ammonium cations. The anionic part can be more variable and include sulfonates, as in CHAPS (3-[(3-Cholamidopropyl)dimethylammonio]-1-propanesulfonate). Other anionic groups are sultaines illustrated by cocamidopropyl hydroxysultaine; betaines, e.g., cocamidopropyl betaine; phosphates, e.g. lecithin. In some embodiments, the surfactant may be a non-ionic surfactant (not charged).

Many long chain alcohols exhibit some surfactant properties, and are provided herein as part of a composition in some embodiments. Prominent among these are the fatty alcohols cetyl alcohol, stearyl alcohol, and cetostearyl alcohol (consisting predominantly of cetyl and stearyl alcohols), and oleyl alcohol. Other surfactants include cocamide MEA, cocamide DEA, dodecyldimethylamine oxide, and polyethoxylated tallow amine (POEA). Examples of non-ionic surfactants include polyoxyethylene glycol alkyl ethers, such as octaethylene glycol monododecyl ether or pentaethylene glycol monododecyl ether; polyoxypropylene glycol alkyl ethers; glucoside alkyl ethers, such as decyl glucoside, lauryl glucoside, or octyl glucoside; polyoxyethylene glycol octylphenol ethers, such as Triton X-100; polyoxyethylene glycol alkylphenol ethers, such as Nonoxynol-9; glycerol alkyl esters, such as glyceryl laurate; polyoxyethylene glycol sorbitan alkyl esters (polysorbate); sorbitan alkyl esters (Spans); block copolymers of polyethylene glycol and polypropylene glycol, or Poloxamers.

In some embodiments, the composition may contain one or more ingredients found in artificial tears in amounts known in the art, including but not limited to: carboxymethyl cellulose, polyvinyl alcohol, hydroxypropyl methylcellulose (a.k.a. HPMC or hypromellose), hydroxypropyl cellulose, hydroxyethyl cellulose (HEC), and hyaluronic acid (a.k.a. hyaluronan, HA), and combinations thereof. In some embodiments, the composition does not contain any of the preceding artificial tear ingredients.

In some embodiments, the surfactant is another peptide or protein. In some embodiments, as a non-limiting example, the surfactant is human serum albumin. In some embodiments, as another non-limiting example, the surfactant is a constrained salt of Lacripep™. In some embodiments, as another non-limiting example, the surfactant is constrained Lacripep™, or a pharmaceutically acceptable salt thereof.

In some embodiments, the surfactant is tyloxapol (formaldehyde oxirane polymer with 4-(2,4,4-trimethylpentan-2-yl)phenol). In an embodiment, the only surfactant is tyloxapol, and no other surfactant agent is present in the composition. In some embodiments the surfactant is DPC. In some embodiments, the only surfactant is DPC, and no other surfactant agent is present in the composition.

In some embodiments, the surfactant, as a non-limiting example tyloxapol, or DPC, is present at between 0.01% and 1%; between 0.05% and 0.9%; 0.1% and 0.8%; 0.2% and 0.7%; 0.3% and 0.6%; 0.4% and 0.5%, or any value contained there between. In some embodiments, the surfactant is present in an amount that is, or is less than, 1%; 0.9%; 0.8%; 0.7%; 0.6%; 0.5%; 0.4%; 0.3%; 0.2%; 0.1%; 0.09%; 0.08%; 0.07%; 0.06%; 0.05%; 0.04%; 0.03%; 0.02%; 0.01%; 0.009%; 0.008%; 0.007%; 0.006%; 0.005%; 0.004%; 0.003%; 0.002%; or 0.001%, or is within a range defined by any two of the preceding values. In some embodiments the surfactant is at a concentration of 0.1 mM to 50 mM, 1 mM to 20 mM, 5 to 15 mM, or any value contained there between. In some embodiments, the surfactant is present in concentration that is, or is less than, 0.1, 0.5, 1, 2, 3, 4, 5, 6, 7, 8, 9, 10, 11, 12, 13, 14, 15, 20, 30, 40, 50 or 100 mM, or is within a range defined by any two of the preceding values.

In some embodiments, the composition does not contain a surfactant, excluding the active peptide, e.g. Lacripep™. In some embodiments, the composition does not contain detectable levels of a surfactant, excluding the active peptide, e.g. Lacripep™.

### Tonicity Agents and Osmolality

In some embodiments, the composition further comprises one or more tonicity agents. Such tonicity agents are in addition to any constrained salt of a polypeptide, constrained peptide, or pharmaceutically acceptable salt thereof, or buffer that has tonicity-modifying effects. In some embodiments, the one or more tonicity agents are selected from propylene glycol, polyethylene glycols, sodium chloride, potassium chloride, magnesium chloride, calcium chloride, simple sugars such as dextrose, fructose, galactose, and/or simple polyols such as the sugar alcohols mannitol, sorbitol, xylitol, lactitol, isomaltitol, maltitol, hydrogenated starch hydrolysates, glycerin, and combinations thereof.

In some embodiments, the one or more tonicity agents are selected from sodium chloride, potassium chloride, magnesium chloride, calcium chloride, dextrose, mannitol, and combinations thereof.

In some embodiments, the tonicity agent is sodium chloride. In some embodiments, the sodium chloride is present at between 0.01% and 1%; between 0.05% and 0.9%; 0.1% and 0.8%; 0.2% and 0.75%; 0.3% and 0.7%; 0.4% and 0.6%; or any value contained there between. In some embodiments, the sodium chloride is present at an amount that is, or is about, 1%; 0.95%; 0.9%; 0.85%; 0.8%; 0.75%; 0.7%; 0.65%; 0.6%; 0.55%; 0.5%; 0.45%; 0.4%; 0.35%; 0.3%; 0.25%; 0.2%; 0.15%; 0.1%; 0.09%; 0.08%; 0.07%; 0.06%; 0.05%; 0.04%; 0.03%; 0.02%; or 0.01%; or is within a range defined by any two of the preceding values.

In some embodiments, the only tonicity agent is sodium chloride, and no other tonicity agent is present in the composition.

In some embodiments, a tonicity agent, as a non-limiting example sodium chloride, is added to the composition to adjust the osmolality to a desired level. In some embodiments, the osmolality of the composition is about 150 to about 400 mOsm/kg; about 170 to about 380 mOsm/kg; about 190 to about 360 mOsm/kg; about 210 to about 340 mOsm/kg; about 230 to about 320 mOsm/kg; about 250 to about 300 mOsm/kg; about 270 to about 280 mOsm/kg; or any value in between. In some embodiments, the osmolality of the composition is about 250 to about 350 mOsm/kg; about 260 to about 340 mOsm/kg; about 270 to about 330 mOsm/kg; about 280 to about 320 mOsm/kg; about 290 to about 310 mOsm/kg; about 150 to about 250 mOsm/kg, or any value in between.

In some embodiments, the osmolality of the composition is, or is about, 150 mOsm/kg; 160 mOsm/kg; 170 mOsm/kg; 180 mOsm/kg; 190 mOsm/kg; 200 mOsm/kg; 210 mOsm/kg; 220 mOsm/kg; 230 mOsm/kg; 240 mOsm/kg; 250 mOsm/kg; 260 mOsm/kg; 270 mOsm/kg; 280 mOsm/kg; 290 mOsm/kg; 300 mOsm/kg; 310 mOsm/kg; 320 mOsm/kg; 330 mOsm/kg; 340 mOsm/kg; or 350 mOsm/kg, or is within a range defined by any two of the preceding values

In some embodiments, the osmolality of the composition is between about 280 mOsm/kg and about 320 mOsm/kg. In one embodiment, the osmolality of the composition is about 300 mOsm/kg. In some embodiments the osmolality of the composition is between 150 and 250 mOsm/kg. In some embodiments, NaCl is used to adjust the osmolality of the solution to the desired level. In an embodiment, the composition is, or is about, isotonic with human tears.

### Solvents

In some embodiments, the composition comprises a solvent. In some embodiments the solvent is added to assist in formulation of the constrained salt of the polypeptide in solution, a clear suspension of a surfactant or a combination of a solution and a clear suspension of a surfactant. In some embodiments, the constrained salt of the polypeptide is prepared in a solvent in a concentrated form, optionally heated to assist in getting the constrained salt of the polypeptide into solution, and the concentrated constrained salt of the polypeptide in solvent is then diluted with other excipients as disclosed herein to prepare the final composition having excipient concentrations disclosed herein. In some embodiments the solvent is a polar aprotic solvent. In some embodiments the polar aprotic solvent is selected from the group consisting of dichloromethane (DCM), N-methylpyrrolidone, tetrahydrofuran (THF), ethyl acetate (EtOAc), acetone, dimethylformamide (DMF), acetonitrile (MeCN), dimethyl sulfoxide (DMSO), propylene carbonate (PC), dimethylacetamide (DMA) and mixtures thereof. In some embodiments the solvent is dimethyl sulfoxide (DMSO). In some embodiments the solvent is a polar protic solvent. In some embodiments the polar protic solvent is selected from the group consisting of formic acid, n-butanol, isopropanol, nitromethane, ethanol, methanol, acetic acid, water and mixtures thereof. In some embodiments the solvent is a mixture of protic and aprotic polar solvents comprising a mixture of one or more of the above solvents. In some embodiments the mixture is water and DMSO. In some embodiments the concentration of solvent in the composition after dilution with other excipients is 0.001% and 10%; between 0.1% and 9%; 0.5% and 8%; 1% and 7%; 0.2% and 3%; 0.4% or 5%, 0.5% and 1.5% or any value contained there between. In some embodiments, the solvent is present in an amount that is, or is less than, 10%; 9%; 8%; 7%; 6%; 5%; 4%; 3%; 2%; 1%; 0.9%; 0.8%; 0.7%; 0.6%; 0.5%; 0.4%; 0.3%; 0.2%; 0.1%; 0.09%; 0.08%; 0.07%; 0.06%; 0.05%; 0.04%; 0.03%; 0.02%; or 0.01%, or is within a range defined by any two of the preceding values.

### Polypeptides and Other Ingredients

In some embodiments, the constrained salt of a polypeptide, constrained polypeptide, or a pharmaceutically acceptable salt thereof, has between 10 to 20 amino acids; between 15 to 30 amino acids; between 20 to 40 amino acids; between 15 to 25 amino acids; or any number contained therein. In some embodiments, the constrained salt of a polypeptide, constrained polypeptide, or a pharmaceutically acceptable salt thereof, has between 10 to 30 amino acids; 11 to 29 amino acids; 12 to 28 amino acids; 13 to 27 amino acids; 14 to 26 amino acids; 15 to 25 amino acids; 16 to 24 amino acids; 17 to 23 amino acids; 18 to 22 amino acids; 19 to 21 amino acids; or any number contained therein. In some embodiments, the constrained salt of a polypeptide, constrained polypeptide, or a pharmaceutically acceptable salt thereof, is, or is about, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids in length, or a range defined by any two of the preceding values.

In some embodiments, the amino acids comprise alpha amino acids. In some embodiments, the amino acids independently comprise natural amino acids, unnatural amino acids, or a combination of any of the foregoing. In some embodiments, each unnatural amino acid independently comprises a crosslinking moiety. In some embodiments, each unnatural amino acid independently comprises a compound of Formula (I).

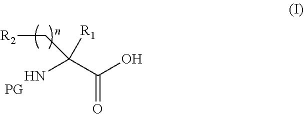

In some embodiments, each n is independently 0, 1, 2, 3, 4, 5, 6, 7, 8, 9, or 10. In some embodiments, PG is a suitable amino-protecting group. In some embodiments, each R₁ is independently hydrogen or methyl. In some embodiments, each R₂ is independently selected from the group consisting of:

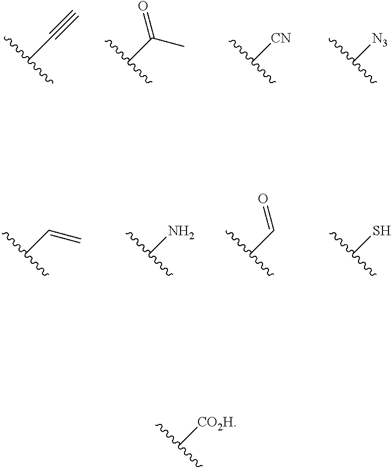

In some embodiments, each unnatural amino acid independently comprises a (D) amino acid, a (L) amino acid, or a combination thereof.

In some embodiments, the C-terminus of the constrained salt of a polypeptide, constrained polypeptide, or a pharmaceutically acceptable salt thereof, is amidated. In some embodiments, the N-terminus of the constrained salt of a polypeptide, constrained polypeptide, or a pharmaceutically acceptable salt thereof, is acetylated. In some embodiments, one or more side chains of the constrained salt of a polypeptide, constrained polypeptide, or a pharmaceutically acceptable salt thereof, are acetylated. In some embodiments, one or more side chains of the constrained salt of a polypeptide, constrained polypeptide, or a pharmaceutically acceptable salt thereof, are amidated. In some embodiments, the N-terminus of the constrained salt of a polypeptide, constrained polypeptide, or a pharmaceutically acceptable salt thereof, is acetylated and the C-terminus of the constrained salt of a polypeptide, constrained polypeptide, or a pharmaceutically acceptable salt thereof, is amidated.

In some embodiments, the constrained salt of a polypeptide comprises, consists or consists essentially of the amino acid sequence: Ac-Lys-Gln-Phe-Ile-Glu-Asn-Gly-Ser-Glu-Phe-Ala-Gln-Lys-Leu-Leu-Lys-Lys-Phe-Ser-Leu-Leu-Lys-Pro-Trp-Ala-NH₂ (SEQ ID NO: 2), where “Ac” represents an N-terminal acetyl group and the C-terminus is amidated (indicated by “NH₂”) In some embodiments, the constrained salt of a polypeptide comprises the amino acid sequence: Ac-Lys-Gln-Phe-Ile-Glu-Asn-Gly-Ser-Glu-Phe-Ala-Gln-Lys-Leu-Leu-Lys-Lys-Phe-Ser-NH₂ (SEQ ID NO: 1), where “Ac” represents an N-terminal acetyl group and the C-terminus is amidated (indicated by “NH₂”). In some embodiments, the polypeptide is a constrained salt, which comprises, consists, or consists essentially of a sequence selected from the group of SEQ ID NOs: 1-9, or fragments, thereof.

In some embodiments, the constrained polypeptide, or a pharmaceutically acceptable salt thereof, comprises, consists or consists essentially of the amino acid sequence: Ac-Lys-Gln-Phe-Ile-Glu-Asn-Gly-Ser-Glu-Phe-Ala-Gln-Lys-Leu-Leu-Lys-Lys-Phe-Ser-Leu-Leu-Lys-Pro-Trp-Ala-NH₂ (SEQ ID NO: 2), where “Ac” represents an N-terminal acetyl group and the C-terminus is amidated (indicated by “NH₂”), and wherein at least two amino acids in the i and i+3, i and i+4, i and i+7 of the helical turn, or a combination of these positions, are replaced or modified with compounds comprising a crosslinking moiety. In some embodiments, the constrained polypeptide, or a pharmaceutically acceptable salt thereof, comprises the amino acid sequence: Ac-Lys-Gln-Phe-Ile-Glu-Asn-Gly-Ser-Glu-Phe-Ala-Gln-Lys-Leu-Leu-Lys-Lys-Phe-Ser-NH₂ (SEQ ID NO: 1), where “Ac” represents an N-terminal acetyl group and the C-terminus is amidated (indicated by “NH₂”), and wherein at least two amino acids in the i and i+3, i and i+4, i and i+7 of the helical turn, or a combination of these positions, are replaced or modified with compounds comprising a crosslinking moiety. In some embodiments, the constrained polypeptide, or a pharmaceutically acceptable salt thereof, comprises, consists, or consists essentially of a sequence selected from the group of SEQ ID NOs: 3-9, or fragments, or pharmaceutically acceptable salts thereof, and wherein at least two amino acids in the i and i+3, i and i+4, i and i+7 of the helical turn, or a combination of these positions, are replaced or modified with compounds comprising a crosslinking moiety. In some embodiments, the polypeptide is a constrained polypeptide, which comprises, consists, or consists essentially of constrained form of a sequence selected from the group of SEQ ID NOs: 1-9, or fragments, thereof.

In some embodiments, the amount of constrained salt of a polypeptide, constrained polypeptide, or a pharmaceutically acceptable salt thereof, in the composition is, or is about, 0.0001% to 1%; 0.0005% to 0.5%; 0.001% to 0.1%; 0.005% to 0.05%; 0.006% to 0.04%; 0.007% to 0.03%; 0.008% to 0.02%; or 0.009% to 0.01%. In some embodiments, the amount of constrained salt of a polypeptide, constrained polypeptide, or a pharmaceutically acceptable salt thereof, in the composition is, or is about, 0.00001% to 0.05%. In an embodiment, the constrained salt of a polypeptide, constrained polypeptide, or a pharmaceutically acceptable salt thereof, is present in the composition at about 0.003% to 0.09% (e.g., 0.005%, 0.01%, 0.02%, 0.03% and ranges thereof). In an embodiment, the constrained salt of a polypeptide, constrained polypeptide, or a pharmaceutically acceptable salt thereof, is present in the composition at about 0.0001% to 0.005% (e.g., 0.005%, 0.001%, 0.0005%, 0.0001%, 0.00001% and ranges thereof).

In some embodiments, the constrained salt of a polypeptide, constrained polypeptide, or a pharmaceutically acceptable salt thereof, is present in the composition in an amount that is, is about, is more than, or is less than, 0.0001, 0.00025, 0.0005, 0.00075, 0.001, 0.002, 0.003, 0.004, 0.005, 0.006, 0.007, 0.008, 0.009, 0.010, 0.011, 0.012, 0.013, 0.014, 0.015, 0.020, 0.030, 0.040, 0.050, 0.060, 0.070, 0.080, 0.090, 0.10, 0.20, 0.30, 0.40, 0.50, 0.60, 0.70, 0.80, 0.90, or 1.0%, or a range defined by any two of the preceding values. In some embodiments, the constrained salt of a polypeptide, constrained polypeptide, or a pharmaceutically acceptable salt thereof, is present in the composition in an amount that is, is about, is more than, or is less than, 0.00001 to 0.0001, or a range defined by any two of the preceding values.

As stated previously, for the percentage (% w/w) value of peptides, constrained salts of peptides, constrained peptides, and pharmaceutically acceptable salts thereof, disclosed herein, the amount (% w/w) is calculated using the molecular weight of the free base form of the unmodified peptide. Thus, the percentage (% w/w) will need to be adjusted when a salt form and/or modified form of the peptide is used if the same molar concentration of the peptide in solution is desired, as is the case in some embodiments herein.

In some embodiments, the composition is a sterile aqueous composition comprising, consisting or consisting essentially of about 0.001% to about 0.05% of a polypeptide, such as a constrained salt of Lacripep™ or the other peptides identified herein, or constrained Lacripep™ or the other peptides identified herein; about 0.001% to about 0.015% anhydrous citric acid; about 0.02% to about 0.40% sodium citrate dihydrate; about 0.0005% to about 0.005% disodium EDTA; about 0.005% to about 0.15% tyloxapol, and optionally, about 0.005% to about 0.1% methylparaben; wherein the pH of the composition is adjusted using NaOH or HCl to be about 6.2 pH to about 6.8 pH, and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.1% to about 1%. In an embodiment, the composition does not include methylparaben. In an embodiment, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents.

In some embodiments, the composition is a sterile aqueous composition comprising, consisting or consisting essentially of about 0.01%±0.001% of a polypeptide, such as a constrained salt of Lacripep™ or the other peptides identified herein, or constrained Lacripep™ or the other peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol, about 0.04%±0.004% methylparaben; wherein the pH of the composition is adjusted using NaOH or HCl to be between about 6.2 to about 6.8. and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.50%±0.05%. In an embodiment, the composition does not include methylparaben.

In some embodiments, the composition is a sterile aqueous composition comprising, consisting or consisting essentially of about 0.005%±0.0005% of a polypeptide, such as a constrained salt of Lacripep™ or the other peptides identified herein, or constrained Lacripep™ or the other peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol, about 0.04%±0.004% methylparaben; wherein the pH of the composition is adjusted using NaOH or HCl to be between about 6.2 to about 6.8. and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.50%±0.05%. In an embodiment, the composition does not include methylparaben.

In some embodiments, the composition is a sterile aqueous composition comprising about 0.001%±0.0001% of a polypeptide, such as a constrained salt of Lacripep™ or the other peptides identified herein, or constrained Lacripep™ or the other peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol, about 0.04%±0.004% methylparaben; wherein the pH of the composition is adjusted using NaOH or HCl to be between about 6.2 to about 6.8. and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.50%±0.05%. In an embodiment, the composition does not include methylparaben.

In some embodiments, the composition is a sterile aqueous composition comprising, consisting or consisting essentially of about 0.0001% to about 0.005% of a polypeptide, such as a constrained salt of Lacripep™ or the other peptides identified herein, or constrained Lacripep™ or the other peptides identified herein; about 0.001% to about 0.015% anhydrous citric acid; about 0.02% to about 0.40% sodium citrate dihydrate; about 0.0005% to about 0.005% disodium EDTA; about 0.005% to about 0.15% tyloxapol, and optionally, about 0.005% to about 0.1% methylparaben; wherein the pH of the composition is adjusted using NaOH or HCl to be about 6.2 pH to about 6.8 pH, and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.1% to about 1%. In an embodiment, the composition does not include methylparaben. In an embodiment, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents.

In some embodiments, including but not limited to the sterile compositions above and herein, the polypeptide is a constrained salt of Lacripep™, having SEQ ID NO: 1. In some embodiments the polypeptide is a constrained salt of a polypeptide having SEQ ID NO: 2. In some embodiments the polypeptide is a constrained salt of a polypeptide having a sequence selected from the group of SEQ ID NOs: 3-9, or a fragment or fragments thereof.

In some embodiments, including but not limited to the sterile compositions above and herein, the polypeptide is a constrained form of Lacripep™, having SEQ ID NO: 1. In some embodiments the polypeptide is a constrained form of a polypeptide having SEQ ID NO: 2. In some embodiments the polypeptide is a constrained form of a polypeptide having a sequence selected from the group of SEQ ID NOs: 3-9, or a fragment or fragments thereof. In some embodiments, including but not limited to the sterile compositions above and herein, the polypeptide is constrained Lacripep™, having SEQ ID NO: 1, wherein at least two amino acids in the i and i+3, i and i+4, i and i+7 of the helical turn, or a combination of these positions, are replaced or modified with compounds comprising a crosslinking moiety, or a pharmaceutically acceptable salt thereof. In some embodiments the polypeptide is a constrained polypeptide having SEQ ID NO: 2, wherein at least two amino acids in the i and i+3, i and i+4, i and i+7 of the helical turn, or a combination of these positions, are replaced or modified with compounds comprising a crosslinking moiety, or a pharmaceutically acceptable salt thereof. In some embodiments the polypeptide is a polypeptide having a sequence selected from the group of SEQ ID NOs: 3-9, or a fragment or fragments thereof, wherein at least two amino acids in the i and i+3, i and i+4, i and i+7 of the helical turn, or a combination of these positions, are or modified with compounds comprising a crosslinking moiety, or a pharmaceutically acceptable salt thereof.

In some embodiments, including but not limited to the sterile compositions above and herein, the pH of the composition is between about 6.5 to about 6.6.

In some embodiments, including but not limited to the sterile compositions above and herein, the osmolality of the composition is between about 280 to about 320 mOsm/kg. In some embodiments, the osmolality of the composition is about 300 mOsm/kg. In some embodiments the osmolality of the composition is between about 150 and about 250 mOsm/kg.

In some embodiments, the composition is a sterile aqueous composition comprising about 0.01%±0.001% constrained salt of a polypeptide, e.g. Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.50%±0.05%.

In some embodiments, the composition is a sterile aqueous composition comprising about 0.005%±0.0005% constrained salt of a polypeptide, e.g., Lacripep™, or the other constrained salts of peptides identified herein constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.50%±0.05%.

In some embodiments, the composition is a sterile aqueous composition comprising about 0.001%±0.0001% constrained salt of a polypeptide, e.g., Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.50%±0.05%.

In some embodiments, the composition is a sterile aqueous composition comprising about 0.0001%±0.00001% constrained salt of a polypeptide, e.g., Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In some embodiments, the amount of NaCl is about 0.50%±0.05%.

In an embodiment, including but not limited to the sterile compositions above and herein, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents. In some embodiments, the amounts of any one or more of the listed ingredients is provided in an amount that is ±5%, and/or ±1% of the listed amount.

In some embodiments, the compositions disclosed herein are prepared as a solution, gel or ointment. Gels or ointments are advantageous in providing the composition in contact with the eye for a longer period of time than a solution or provide other benefits. Therefore, in one embodiment, a gel or ointment is useful when applying the composition to the subject when the subject will be sleeping, or when the subject's eyes will be closed for an extended period of time (e.g., 1, 2, 3, 4, 5 or more hours). Gels or ointments may be used at other times based on user preference.

### Other Therapeutic Ingredients

In some embodiments the compositions include one or more additional therapeutic agents in addition to the constrained salts of polypeptides or constrained polypeptides disclosed herein. These therapeutic agents can include substances known to those skilled in the art for the treatment of dry eye and related syndromes and conditions, including Sjögren's Syndrome. The additional therapeutic ingredients can treat the disease, syndrome or condition, or can relieve symptoms associated with the disease, syndrome or condition. A non-exhaustive list of additional therapeutic agents includes: cholinergics (e.g., pilocarpine, cevimeline), Cyclosporine, Lifitegrast, Dexamethasone (or other cortico-steroids such as prednisolone), Hyaluronic acid (and its derivatives) with or without chondroitin sulfate, Cyclokat, SI-614, skQ1, Cis-UCA, CycloASol, RGN-259, Diquafosol, Anakinra, Tofacitinib, EBI-005, EGP-437, KP-121, MIM-D3, OTX-DP, rebamipide (OPC-12759), and RU-101. In some embodiments, the one or more additional therapeutic agents are provided as a salt of the polypeptide. Artificial tears and other lubricants that contain one or more of carboxymethyl cellulose, polyvinyl alcohol, hydroxypropyl methylcellulose (a.k.a. HPMC or hypromellose), hydroxypropyl cellulose, ethylene glycol polymers, and hyaluronic acid (a.k.a. hyaluronan, HA), and tear ointments such as white petrolatum, mineral oil, and similar lubricants can also be included in the compositions. These additional therapeutic agents can be included in known therapeutic amounts.

### Containers and Kits

In some embodiments, the composition is provided in a kit comprising one or more multi-use containers. In some embodiments, the multi-use container comprises a protective cap and a liquid storage bottle, wherein the cap is connected to the bottle via a flexible connector. A blocking plug is arranged in the middle of the top surface of the protective cap. A conical, or other suitable shape, liquid outlet is arranged in the middle of the bottle cover and is tightly matched with the blocking plug of the protective cap. Thus, the sterile composition may be placed into the container for multiple uses.

In some embodiments, the amount of the composition in the container is, or is about: 0.1-0.5, 0.5-1.0, 1-2, 2-5, 5-10, 10-20, 20-30, or 30-60 mL or ranges in between. Containers may be bottles, tubes, vials or other suitable containers. Multi-use containers may be accompanied by instructions to use for a 12 hour, 24 hour, 2-7 day cycle, one month cycle or until a stated expiration date. A single-use container may be suitable for use in one eye or both eyes for a single application cycle.

In some embodiments, the composition is provided in a in a kit comprising a single-use container. In some embodiments, the composition is provided in a in a kit comprising a plurality of single-use containers. In some embodiments, the single-use container comprises a vessel for holding liquid, a removable seal top for sealing the vessel, and, optionally, a neck portion interconnecting the vessel and the seal top. Kits comprises multiple single-use containers along with instructions to use are provided in several embodiments.

In some embodiments, the container comprises a pharmaceutically inert material. In some embodiments, the container comprises glass, polyvinyl chloride, polypropylene, polyethylene terephthalate, polyethylene terephthalate, polyethylene terephthalate G, high-density polyethylene, low-density polyethylene, polybutylene terephthalate, polyurethane, polyethylene vinyl acetate, silicone, acrylonitrile butadiene styrene, polytetrafluoroethylene, polycarbonate, polystyrene, polymethylmethacrylate, polysulfone, polyvinylidene chloride, or combinations thereof.

In some embodiments, the container comprises polyvinyl chloride, polypropylene, low-density polyethylene, polyurethane, polyethylene vinyl acetate, silicone, or combinations thereof.

In some embodiments, the amount of composition in the container is, or is about, 0.02 mL; 0.05 mL to 1 mL; 0.1 mL to 0.95 mL; 0.15 mL to 0.8 mL; 0.2 mL to 0.85 mL; 0.25 mL to 0.8 mL; 0.3 mL to 0.75 mL; 0.35 mL to 0.7 mL; 0.4 mL to 0.65 mL; 0.45 mL to 0.6 mL; 0.5 mL to 0.55 mL; or any amount in between.

In some embodiments, the amount of composition in the container is, or is about, 0.02 mL; 0.025 mL; 0.030 mL; 0.035 mL; 0.040 mL; 0.045 mL; 0.050 mL; 0.055 mL; 0.060 mL; 0.065 mL; 0.070 mL; 0.075 mL; 0.1 mL; 0.15 mL; 0.2 mL; 0.25 mL; 0.3 mL; 0.35 mL; 0.4 mL; 0.45 mL; 0.5 mL; 0.55 mL; 0.6 mL; 0.65 mL; 0.7 mL; 0.75 mL; 0.8 mL; 0.85 mL; 0.9 mL; 0.95 mL; or 1 mL of the composition, or an amount that is within a range defined by any two of the preceding values.

### Ophthalmic and Other Administration

In some embodiments, the composition is administered topically to the eye. In some embodiments, the composition is administered to an individual suffering from any form of dry eye, or dry eye (or other symptoms, such as dry mouth) associated with Sjögren's Syndrome, for the treatment thereof. In some embodiments it is administered as an oral rinse, tab, patch, spray or lozenge to the mouth. The compositions described herein can be provided as liquids (solutions, gels, ointments etc.) or in other suitable forms, such as powders or on patches, tabs, etc. In some embodiments, the compositions described herein are used to achieve one or more of the following: restore basal tearing, salivation, general mucosal and ocular surface wetness; restore ocular surface and mucosal homeostasis, rapidly but transiently promote autophagy to eliminate pressure, stress or degenerative disease throughout the eye and in other organs; reduce inflammation, promote wound healing (such as corneal post refractive surgery or oral wound healing), stabilize the tear lipid layer and suppress bacterial infection.

In some embodiments, administration topically to the eye comprises administering one or more drops of the composition to the surface of the eye. For example, in one embodiment, a user is instructed to apply to the eye surface, and not to a contact lens). In other embodiments, the drops (or other application) is suitable for administration while wearing contact lenses. In some embodiments, the composition is administered from the container as a single drop delivered as a single dose to each eye. In some embodiments, the drop is about 0.020 mL to about 0.050 mL, or any volume in between. In some embodiments, the drop is about 0.035 mL.

In some embodiments, the administration of the composition to the eye improves one or more symptoms clinical signs or measures of dry eye or Sjögren's Syndrome. Improvements in dry eye clinical signs can be assessed by one or more of the following:
Fluorescein corneal staining (FCS) (0 to 3 scale by region, total 0-15 scale, using the NEI/Industry Workshop scale) with particular attention paid to the inferior corneal region.
Lissamine green conjunctival staining (LGCS) (0 to 3 scale by region, total 0-18 scale, using NEI/Industry Workshop scale)
Anesthetized Schirmer test (mm of wetting in 5 minutes),
Tear film break-up time (number of seconds)
Dry eye-related ocular symptoms questionnaire (SANDE: how frequent and how severe are dry eye symptoms).

In some embodiments, the composition is a sterile aqueous composition comprising, consisting or consisting essentially of about 0.001% to about 0.05% of constrained salt of a polypeptide, e.g., Lacripep™, or the other constrained salts of peptides identified herein, constrained Lacripep™, or the other constrained peptides identified herein; about 0.001% to about 0.015% anhydrous citric acid; about 0.02% to about 0.40% sodium citrate dihydrate; about 0.0005% to about 0.005% disodium EDTA; about 0.005% to about 0.15% tyloxapol, and about 0.005% to about 0.1% methylparaben; wherein the pH of the composition is adjusted using NaOH or HCl to be about 6.2 pH to about 6.8 pH, and the osmolality of the composition is adjusted using NaCl to be between about 250 to about 350 mOsm/kg. In some embodiments, the amount of NaCl is about 0.1% to about 1%. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In an embodiment, the composition does not include methylparaben. In an embodiment, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents.

In some embodiments, the composition is a sterile aqueous composition comprising, consisting or consisting essentially of about 0.001% to about 0.05% of constrained salt of a polypeptide, e.g., Lacripep™, or the other constrained salts of peptides identified herein, constrained Lacripep™, or the other constrained peptides identified herein; about 0.001% to about 0.015% anhydrous citric acid; about 0.02% to about 0.40% sodium citrate dihydrate; about 0.0005% to about 0.005% disodium EDTA; and about 0.005% to about 0.15% tyloxapol; wherein the pH of the composition is adjusted using NaOH or HCl to be about 6.2 pH to about 6.8 pH, and the osmolality of the composition is adjusted using NaCl to be between about 250 to about 350 mOsm/kg. In some embodiments, the amount of NaCl is about 0.1% to about 1%. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO. In an embodiment, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents.

In some embodiments, the composition is a sterile aqueous composition comprising about 0.01%±0.001% of constrained salt of a polypeptide, e.g., Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments, the amount of NaCl is about 0.50%±0.05%. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO.

In some embodiments, the composition is a sterile aqueous composition comprising about 0.005%±0.0005% constrained salt of a polypeptide, e.g., Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments, the amount of NaCl is about 0.50%±0.05%. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO.

In some embodiments, the composition is a sterile aqueous composition comprising about 0.001%±0.0001% constrained salt of a polypeptide, e.g., Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments, the amount of NaCl is about 0.50%±0.05%. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO.

In some embodiments, the composition is a sterile aqueous composition comprising about 0.0001%±0.00001% constrained salt of a polypeptide, e.g., Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol; wherein the pH of the composition is adjusted using NaOH or HCl to be between about 6.2 to about 6.8, and the osmolality of the composition is adjusted using NaCl to be between about 250 to about 350 mOsm/kg. In some embodiments, the amount of NaCl is about 0.50%±0.05%. In some embodiments the osmolality of the composition is adjusted using NaCl to be between about 150 to about 250 mOsm/kg. In some embodiments the composition comprises DPC, and/or another surfactant instead of or in addition to tyloxapol. In some embodiments the composition comprises a solvent. In some embodiments the solvent is present in an amount of about 1%. In some embodiments the solvent is DMSO.

In some embodiments, the composition is a sterile aqueous composition comprising about 0.001%±0.0001% constrained salt of a polypeptide, e.g., Lacripep™, or the other constrained salts of peptides identified herein, constrained Lacripep™, or the other constrained peptides identified herein; about 0.0098%±0.001% anhydrous citric acid; about 0.279%±0.028% sodium citrate dihydrate; about 0.001%±0.0001% disodium EDTA; about 0.05%±0.005% tyloxapol or about 0.10%±0.01% tyloxapol; about 1.0%±0.1% DMSO (dimethyl sulfoxide); wherein the pH of the composition is adjusted using NaOH or HCl to be between about 6.2 to about 6.8, and the osmolality of the composition is adjusted using NaCl to be between about 250 to about 350 mOsm/kg. In some embodiments the osmolality of the composition is adjusted using NaCl to be between about 150 to about 250 mOsm/kg.

In some embodiments the composition is prepared by adding the Lacripep Fatty Acid Salt API (constrained salt) into a glass beaker at 0.001%. DMSO is added at 1% onto the API. Excipient buffers are made with all other excipients at 10 times their final concentration (before dilution). Buffer solutions and API/DMSO product are heated to ˜60° C. Buffer is slowly added to the API/DMSO up to 10% of the batch. The batch is then brought up to 100% with water, bringing the concentration of excipients down to their final concentrations. In some embodiments the constrained fatty acid salt of the peptide (e.g. Lacripep™) is palmitic acid salt or linoleic acid salt.

In some embodiments, the composition is a sterile aqueous composition comprising a formulation disclosed in Table 1. In some embodiments the composition is prepared as described in Example 4.

### EXAMPLES

The following are non-limiting examples of some of the embodiments described herein.

### Example 1—Constrained Oleic Acid Salt of Lacripep™

Lacripep™ is synthesized using standard solid-phase peptide synthesis techniques. Any remaining protecting groups are removed, the peptide is cleaved from the resin, purified by precipitation from diethyl ether and further purified by HPLC containing 0.1% acetic acid in the mobile phase. The Lacripep™ containing fractions are frozen and lyophilized to yield the acetate salt of Lacripep™.

The purified acetate salt of the peptide is suspended in water:acetonitrile mixture (4:1) at room temperature. Four equivalents of oleic acid (one per lysine sidechain) are added and the mixture is gently stirred at room temperature for five minutes. The mixture is frozen and lyophilized to a powder. The lyophilized powder is suspended in water and the pH is adjusted to between 6.2 to 6.8 with citric acid and sodium citrate and the osmolality to between 250 to 350 mOsm/kg. The stabilized (constrained) oleic acid salt of Lacripep™ is characterized using known techniques, for example, mass spectrometry, and circular dichroism.

### Example 2—Constrained Dodecylphosphocholine Form of Lacripep™ Vs Acetate Salt of Lacripep™ in Phosphate Buffer

Lacripep™ (SEQ ID NO:1) was synthesized either without (unmodified, KQFIENGSEFAQKLLKKFS) or with (modified, Ac-KQFIENGSEFAQKLLKKFS-NH₂)N-terminal acetylation and C-terminal amidation as an acetate salt. The compounds (0.2 mg/ml) were reconstituted in 10 mM phosphate, 137 mM NaCl, 2.7 mM KCl buffer, pH 7.4 with 10 mM dodecylphosphocholine for circular dichroism. CD spectra were obtained using the Jasco 810 CD/ORD with Fluorescence Monochrometer in a 1 mm quartz cell from Starna Cells, Inc. Spectra were obtained at 35° C. from 250 nm to 190 nm in continuous scanning mode (scanning speed, 100 nm/min; data pitch, 0.1 nm; bandwidth, 1 nm; response time, 4 s) with a nitrogen flow rate of 100 mL/min. An average of 3 spectra were obtained for each sample (N=3). Analysis of the dichroism spectra can use methods known in the art, including, DichroWeb: On-line analysis for protein Circular Dichroism spectra; Whitmore, L. and Wallace, B. A. (2008) Biopolymers 89: 392-400; Whitmore, L. and Wallace, B. A. (2004) Nucleic Acids Research 32: W668-673, each of which is incorporated by reference herein in its entirety.

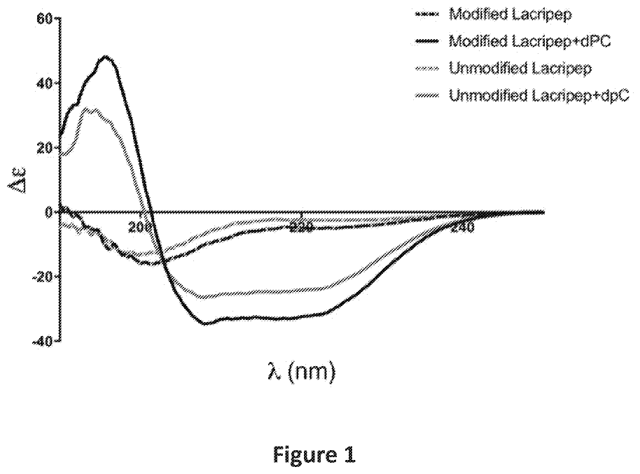

FIG. 1 shows the results of the CD spectra. As expected, both the unmodified and modified acetate salt forms showed little dichroism, indicating that it had little alpha helicity (27% and 28%, respectively). In contrast, both the unmodified and terminally modified dodecylphosphocholine (10 mM dpC, aka DPC) forms showed high levels of dichroism, indicating alpha helical structure, with the modified dpC form having more helicity than the unmodified dpC form (respectively 100% with 0% irregular vs 92% with 8% irregular).

### Example 3—Constrained Oleic Acid Salt of Lacripep™ Vs Acetate Salt of Lacripep™ in Citrate Buffer

Lacripep™ (SEQ ID NO:1) was synthesized either without or with N-terminal acetylation and C-terminal amidation as an acetate salt. The compounds were reconstituted in 10 mM sodium citrate buffer, pH 6.5 for circular dichroism. Lacripep™ synthesized either without or with N-terminal acetylation and C-terminal amidation as an acetate salt was converted to an oleate salt by dissolving 1 equivalent of the Lacripep™ acetate salt and 4 equivalents of oleic acid (one oleic acid per lysine residue in Lacripep™) in a mixture of water buffered with citric acid to pH 6.5. The resulting solutions were then flash frozen and lyophilized under vacuum to remove the more volatile acetic acid. The lyophilized powder is suspended in water and the pH is adjusted to between 6.2 to 6.8 with citric acid and sodium citrate and the osmolality to between 250 to 350 mOsm/kg. The four resulting citric acid buffered solutions, (acetate salt, unmodified, without terminal modification, acetate salt modified with terminal modification, oleate salt without terminal modification, oleate salt with terminal modification) were tested for circular dichroism.

For circular dichroism, all samples used had a concentration of 0.2 mg/mL. CD spectra were obtained using the Jasco 810 CD/ORD with Fluorescence Monochrometer in a 1 mm quartz cell from Starna Cells, Inc. Spectra were obtained at 35° C. from 250 nm to 190 nm in continuous scanning mode (scanning speed, 100 nm/min; data pitch, 0.1 nm; bandwidth, 1 nm; response time, 4 s) with a nitrogen flow rate of 100 mL/min. An average of 3 spectra were obtained for each sample (N=3).

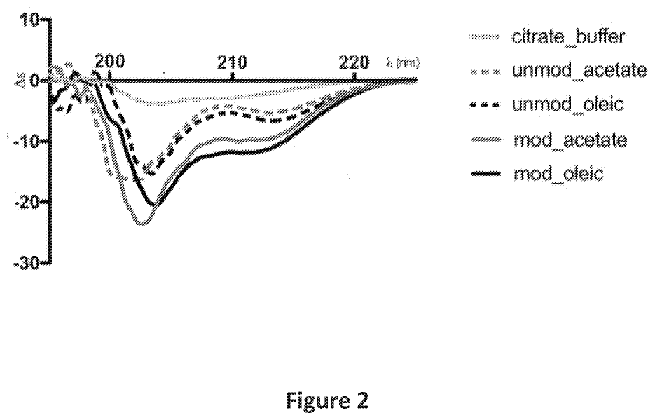

FIG. 2 shows the results of the CD spectra. The terminally modified oleate salt form showed the highest highest level of alpha helicity (57%) as demonstrated by the strongest negative change in ellipticity in the CD spectra. The unmodified oleate salt form was next (38% helicity), followed by the modified acetate salt form (29% helicity), and the unmodified acetate salt form last (28% helictiy). When compared to the results of Example 2, FIG. 1, it appears that citrate buffer slightly enhances the stability of the alpha helix in comparison to phosphate buffer for both the unmodified and terminally modified acetate salt forms.

### Example 4—Constrained Fatty Acid Salt of Lacripep™ in DMSO with Concentrated Buffer Solution

Examples of formulations of Lacripep™ Fatty Acid Salt with improved solubility include the following formulations.

Procedure: Batches were produced at 100 g scale. Lacripep™ Fatty Acid Salt API was added into a glass beaker at 0.001%. DMSO was added at 1% onto the API. Two tyloxapol/excipient buffer concentrations were made with tyloxapol at 0.5% and 1%; all other excipients were at 10 times their final concentration (before dilution). Buffer solutions and API/DMSO product was heated to ˜60° C. Buffer was slowly added to the API/DMSO up to 10% of the batch. The batch was then brought up to 100% with water, bringing the concentration of excipients down to their final concentrations.

TABLE 1

| | | Buffer | Buffer | | | | |
|---|---|---|---|---|---|---|---|
| | | System | System | | | Final | Final |
| | | 10X; | 10X; | Palmitic | Linoleic | Concentrations | Concentrations |
| | | Tyloxapol | Tyloxapol | Acid | Acid | Palmitic Acid | Linoleic Acid |
| | | at 0.5% | at 1.0% | Salt | Salt | Salt Batches | Salt Batches |
| | Ingredient | % w/w | % w/w | % w/w | % w/w | % w/w | % w/w |
| Phase | Sodium Citrate, | 2.79 | 2.79 | — | — | 0.279 | 0.279 |
| A | Anhydrous | | | | | | |
| Buffer | Citric Acid, | 0.098 | 0.098 | — | — | 0.0098 | 0.0098 |
| | Anhydrous | | | | | | |
| | Edetate | 0.01 | 0.01 | — | — | 0.001 | 0.001 |
| | Disodium | | | | | | |
| | Sodium | 5.0 | 5.0 | — | — | 0.50 | 0.50 |
| | Chloride | | | | | | |
| | Purified Water | 91.602 | 91.602 | — | — | 9.16 | 9.16 |
| | Tyloxapol | 0.5 | 1.0 | — | — | 0.05 / 0.10 | 0.05 / 0.10 |
| Batches | LacriPep API | — | — | 0.001 | 0.001 | 0.001 | 0.001 |
| | DMSO | — | — | 1.0 | 1.0 | 1.0 | 1.0 |
| | Phase A Buffer | — | — | 10.0 | 10.0 | — | — |
| | Q.S. w/ | — | — | ~89 | ~89 | ~89 | ~89 |
| | Purified Water | | | | | | |

TABLE 2
Results:

| | Palmitic Acid | Palmitic Acid | Linoleic Acid | Linoleic Acid |
|---|---|---|---|---|
| | Salt with 0.05% | Salt with 0.1% | Salt with 0.05% | Salt with 0.1% |
| | Tyloxapol | Tyloxapol | Tyloxapol | Tyloxapol |
| API appearance in | Mostly fine powder | Fine powder with | Fine powder with | Mostly fine powder |
| vessel | with few larger | no larger particles. | no larger particles. | with few larger |
| | particles. | | | particles. |
| Did API dissolve | Nearly soluble. | Nearly soluble. | Mostly soluble. | Somewhat soluble. |
| in DMSO? | Most API dissolved, | Most API dissolved, | Most API dissolved, | Most API dissolved, |
| | very few particles | graininess on sides | graininess on sides | several small particles |
| | present in liquid, | of vessel. | of vessel. | present in liquid. |
| | graininess on | | | |
| | sides of vessel. | | | |
| Tyloxapol/excipients | Cloudy precipitate | API stayed in | API stayed in | Cloudy precipitate |
| addition observations | formed then dissolved. | solution, clear | solution, clear | formed then dissolved. |
| | API stayed in solution; | solution. | solution. | API went more into |
| | clear solution with | | | solution with only |
| | very few large | | | small amounts of |
| | particles present. | | | graininess on sides. |
| Final appearance | Clear solution with | Clear solution. | Clear solution. | Clear solution with |
| after water added | very few large | | | very few large |
| to Q.S. batch | particles present. | | | particles present. |

Conclusion: API dissolved in the DMSO but appeared to be particle size dependent as larger agglomerations had more difficulty dissolving. The increased buffer concentration appeared to improve the dissolution of API, even after Q.S. Tyloxapol at 0.10% appeared to work comparably if not better than 0.05%

### Example 5—Disulfide Constrained Lacripep™

A peptide having the sequence KQCIENCSEFAQKLLKKFS is synthesized using standard solid-phase peptide synthesis techniques. Any protecting groups are removed, the peptide is cleaved from the resin, and then purified using HPLC or precipitation from diethyl ether.

The peptide is suspended in 50% acetic acid (aq.) and 1 M HCl is added. 0.1 M iodine in acetic acid is added drop-wise and the reaction is stirred at room temperature for three hours. The reaction is then quenched with drop-wise addition of 1 M sodium thiosulfate (aq.). The solvent is removed under reduced pressure and the resulting residue purified by ether precipitation followed by HPLC to provide the disulfide-constrained peptide.

In the alternative, the peptide is suspended in 1.0 M acetic acid (aq.) and oxygen is bubbled through the solution for 3 hours at room temperature, followed by removal of the solvent under reduced pressure, and purification by ether precipitation and HPLC to provide the disulfide-constrained peptide. The disulfide-constrained peptide is characterized using known techniques, for example, mass spectrometry, circular dichroism, and NMR spectroscopy.

### Example 6—Alkene Constrained Lacripep™

A peptide having the sequence KQFIENXSEFAQKXLKKFS is synthesized using standard solid-phase peptide synthesis techniques. Each X is 2-amino-8-nonenoic acid. The solvent is removed from the fully-protected peptide-bund resin, which is re-suspended in 1,2-dichloroethane (DCE) at room temperature. Grubbs' Second Generation ring-closing metathesis (RCM) catalyst is added, and the reaction is agitated at room temperature for 3 hours. An analytical sample of the peptide is cleaved and analyzed by mass spectrometry. If the conversion is less than 95%, the solvent is removed, the resin is washed with DCE, the resin is resuspended in DCE, and a second round of RCM is performed. If a faster reaction time is needed, the reaction is performed in dichlorobenzene and pulsed 10-20× at up to 120-200° C. in a microwave reactor.

Any remaining protecting groups are removed under standard conditions, and the solvent is removed under reduced pressure. The resulting residue purified by ether precipitation followed by HPLC to provide the alkene-constrained peptide. The alkene-constrained peptide is characterized using known techniques, for example, mass spectrometry, circular dichroism, and NMR spectroscopy.

### Example 7—Alkene Constrained Lacripep™ Acetate Salts in Citrate Buffer

Alkene Constrained Analogs of Lacripep™ (SEQ ID NO:1) were synthesized with N-terminal acetylation and C-terminal amidation as an acetate salt. The compounds were reconstituted in 10 mM sodium citrate buffer, pH 6.5 for circular dichroism, with or without 10 mM n-DodecylPhosphoCholine (DPC). The resulting citric acid buffered solutions were tested for circular dichroism.

For circular dichroism, all samples used had a concentration of 0.2 mg/mL. CD spectra were obtained using the Jasco 810 CD/ORD with Fluorescence Monochrometer in a 1 mm quartz cell from Starna Cells, Inc. Spectra were obtained at 35° C. from 250 nm to 190 nm in continuous scanning mode (scanning speed, 100 nm/min; data pitch, 0.1 nm; bandwidth, 1 nm; response time, 4 s) with a nitrogen flow rate of 100 mL/min. An average of 3 spectra were obtained for each sample (N=3).

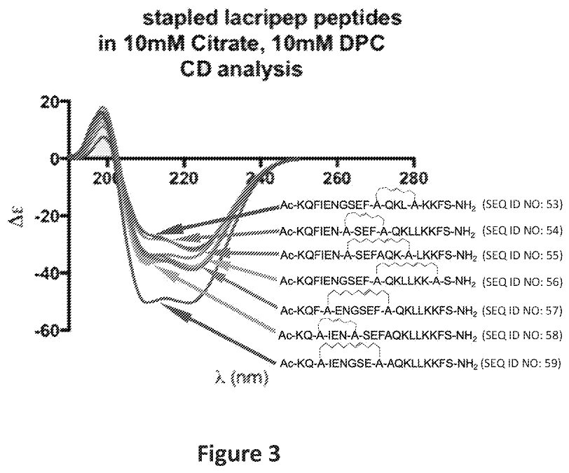

FIG. 3 shows the results of the CD spectra in the presence of 10 mM DPC. The i-i+7 stapled peptide, derived from Ac-KQ [2-(7-octenyl) alanine]-IENGSE-[2-(4-pentenyl) alanine]-AQKLLKKFS-NH₂ (SEQ ID NO: 59) according to the method of Example 6

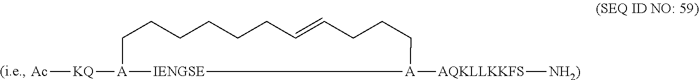

exhibited the most intensely negative CD signal (approximately −50 Δε) between 200 nm and 230 nm indicating the highest helical content. Whereas the i-i+4 stapled peptides derived from Ac-KQFIEN [2-(4-octenyl) alanine]-SEF-[2-(4-pentenyl) alanine]-QKLLKKFS-NH₂ (SEQ ID NO: 54) and Ac-KQFIENGSEF [2-(4-octenyl) alanine]-QKL-[2-(4-pentenyl) alanine]-KKFS—NH₂ (SEQ ID NO: 53) exhibited the least intense negative CD signals (approximately −20 to −30 Δε) between 200 nm and 230 nm indicating the lowest helical content. The remaining i-i+7 stapled peptides derived from (Ac-KQF [2-(7-octenyl) alanine]-ENGSEF-[2-(4-pentenyl) alanine]-QKLLKKFS-NH₂ (SEQ ID NO: 57), Ac-KQFIEN [2-(7-octenyl) alanine]-SEFAQK-[2-(4-pentenyl) alanine]-LKKFS-NH₂ (SEQ ID NO: 55), and Ac-KQFIENGSEF [2-(7-octenyl) alanine]-QKLLKK-[2-(4-pentenyl) alanine]-S-NH₂) (SEQ ID NO: 56) and the i-i+4 stapled peptides (Ac-KQ [2-(4-octenyl) alanine]-IEN-[2-(4-pentenyl) alanine]-SEFAQKLLKKFS-NH₂ (SEQ ID NO: 58), Ac-KQF [2-(4-octenyl) alanine]-ENG-[2-(4-pentenyl) alanine]-EFAQKLLKKFS-NH₂ (SEQ ID NO: 61) stapled, i-i+4, and Ac-KQFIENGSEFAQK [2-(4-octenyl) alanine]-LKK-[2-(4-pentenyl) alanine]-S—NH₂) (SEQ ID NO: 62) exhibited intermediate helicity with negative CD signals of between −30 and −40 Δε between 200 nm and 230 nm.

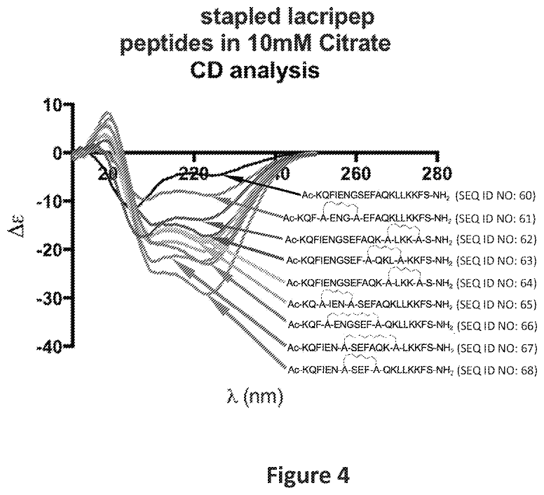

FIG. 4 shows the results of the CD spectra in the absence of 10 mM DPC. In the absence of surfactant (DPC), the Lacripep peptide exhibits little helicity whereas the stapled peptides show more substantial helicity.

### Example 8—Cysteine Disulfide Constrained Lacripep™ Acetate Salts in Citrate Buffer

Cysteine Disulfide Constrained Analogs of Lacripep™ (SEQ ID NO:1) were synthesized with N-terminal acetylation and C-terminal amidation as an acetate salt. The compounds were reconstituted in 10 mM sodium citrate buffer, pH 6.5 for circular dichroism, with or without 10 mM DPC. The resulting citric acid buffered solutions were tested for circular dichroism.

For circular dichroism, all samples used had a concentration of 0.2 mg/mL. CD spectra were obtained using the Jasco 810 CD/ORD with Fluorescence Monochrometer in a 1 mm quartz cell from Starna Cells, Inc. Spectra were obtained at 35° C. from 250 nm to 190 nm in continuous scanning mode (scanning speed, 100 nm/min; data pitch, 0.1 nm; bandwidth, 1 nm; response time, 4 s) with a nitrogen flow rate of 100 mL/min. An average of 3 spectra were obtained for each sample (N=3).

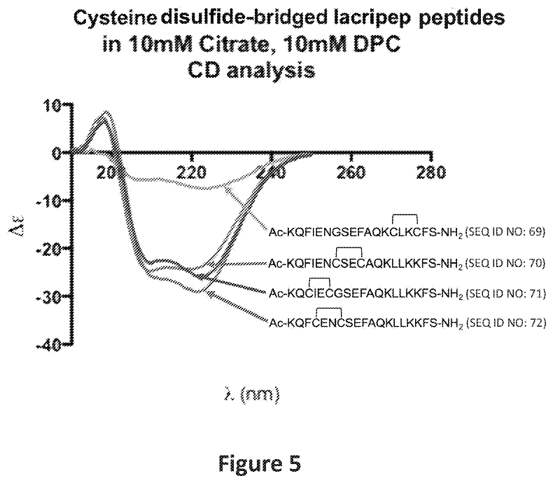

FIG. 5 shows the results of the CD spectra in the presence of DPC. The Cysteine Disulfide i-i+2 constrained peptide, Ac-KQF C(S-)EN C(S-)SE FAQ KLL KKF S-NH₂ (SEQ ID NO: 72) exhibited the most intensely negative CD signal (approximately −20 to −30 Δε) between 200 nm and 230 nm indicating the highest helical content. Whereas the Cysteine disulfide i-i+2 constrained peptide, Ac-KQF IEN GSE FAQ KC(S-)L KC(S-)F S-NH₂ (SEQ ID NO: 69), exhibited the least intense negative CD signal (approximately −5 to −10 Δε) between 200 nm and 230 nm indicating the lowest helical content. The remaining Cysteine Disulfide i-i+2 constrained peptides, Ac-KQF C(S-)EN C(S-)SE FAQ KLL KKF S-NH₂ (SEQ ID NO: 72), and Ac-KQF IEN C(S-)SE C(S-)AQ KLL KKF S-NH₂ (SEQ ID NO: 70), exhibited intermediate strength negative CD signals (approximately −20 to −25 Δε) between 200 nm and 230 nM and thus intermediate helicity.

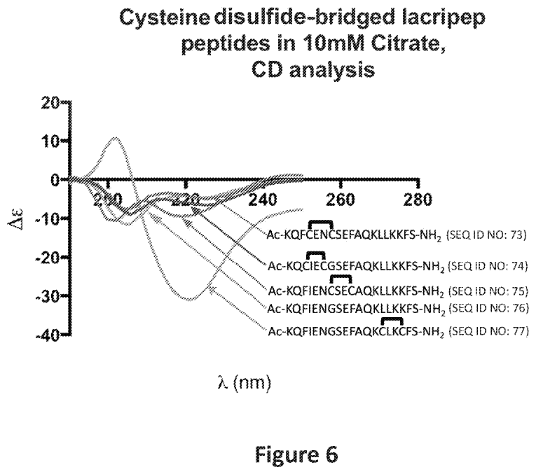

FIG. 6 shows the results of the CD spectra for i-i+2 constrained peptides and lacripep in the absence of 10 mM DPC. The i-i+2 disulfide constrained peptides (i.e., Ac-KQF C(S-)EN C(S-)SE FAQ KLL KKF S-NH₂ (SEQ ID NO: 73), Ac-KQF IEN GSE FAQ KC(S-)L KC(S-)F S-NH (SEQ ID NO: 77)₂, Ac-KQF C(S-)EN C(S-)SE FAQ KLL KKF S-NH₂ (SEQ ID NO: 73), and Ac-KQF IEN C(S-)SE C(S-)AQ KLL KKF S-NH₂ (SEQ ID NO: 75)) and lacripep show significant helicity in the absence of DPC surfactant. Overall the disulfide constrained peptides and lacripep behave similarly with regard to helicity in the presence and absence of DPC. In some embodiments, the presence of DPC is included in formulation to preserve helicity.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the embodiments of the invention(s).

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

The indefinite article “a” or “an” does not exclude a plurality. The use of “about” before a number includes the number itself. For example, “about 5” provides express support for “5”.

1. A constrained salt of a peptide comprising a sequence selected from SEQ ID NOs: 1-9, wherein the salt comprises a phospholipid, a straight-chain fatty acid, a branched-chain fatty acid, or a combination of any of the foregoing, and wherein at least 90% of the peptide is in an alpha helical conformation.

2. (canceled)

3. The constrained salt of claim 1, wherein the salt is a straight-chain fatty acid.

4. A peptide comprising SEQ ID NO: 1 or 2, or a pharmaceutically acceptable salt thereof, wherein at least two amino acids at the i and i+4 or i and i+7 positions of a helical turn are replaced or modified with a compound comprising a crosslinking moiety and said crosslinking moieties are covalently bonded, and wherein said peptide has an alpha helical conformation.

5-12. (canceled)

13. A liquid composition comprising:
0.00001-0.05% of the constrained salt of a peptide of claim 1, wherein the amount of peptide is weight/weight percentage calculated using the molecular weight of the free base and/or unconstrained form of the peptide;
0.01-0.6% of a buffer;
0.0005-0.01% disodium EDTA;
and sodium chloride;
wherein the pH of the composition is between 6.2 to about 6.8 and the osmolality of the composition is between about 150 to 350 mOsm/kg, and wherein at least 90% of the peptide is in an alpha helical conformation.

14. (canceled)

15. The composition of claim 13, further comprising 0.01-0.1% tyloxapol.

16. The composition of claim 13, wherein the amount of the constrained salt of the peptide is 0.01%, 0.005%, 0.001%, 0.00025%, 0.0001% or 0.00001%, wherein the amount of peptide is weight/weight percentage calculated using the molecular weight of the free base and/or unconstrained form of the peptide,
wherein the peptide consists of SEQ ID NO: 1, wherein the N-terminus is acetylated and the C-terminus is amidated.

17. The composition of claim 13, wherein the buffer is a citrate buffer.

18. The composition of claim 17, wherein the citrate buffer comprises 0.0098% anhydrous citric acid and 0.279% sodium citrate dihydrate.

19. The composition of claim 13, wherein the pH of the composition is about 6.5.

20. The composition of claim 13, wherein the osmolality of the composition is between 150 to 250 mOsm/kg.

21. (canceled)

22. The composition of claim 13, wherein the amount of NaCl is between 0.4% and 0.6%.

23. The composition of claim 13, wherein the amount of NaCl is 0.5%.

24. (canceled)

25. The composition of claim 13, wherein the composition is sterile.

26-27. (canceled)

28. A kit, comprising a plurality of single-use containers, wherein each container comprises a vessel for holding the composition of claim 13.

29-32. (canceled)

33. A method of administration comprising topically applying one or more drops of the composition of claim 13 to an eye.

34. (canceled)

35. The method of claim 33, wherein the administration is for the treatment of dry eye.

36. The method of claim 33, wherein the administration is for the treatment of one or more symptoms or signs of Sjögren's Syndrome.

37. (canceled)

38. The constrained salt of a peptide of claim 1, wherein the phospholipid, the straight-chain fatty acid, the branched-chain fatty acid, or the combination of any of the foregoing, is selected from salts of myristoleic acid, palmitoleic acid, sapienic acid, oleic acid, elaidic acid, vaccenic acid, linoleic acid, linolaidic acid, alpha-linolaidic acid, arachidonic acid, eicopentaenoic acid, eruric acid, docosahexaenoic acid, caprylic acid, capric acid, lauric acid, myristic acid, palmitic acid, stearic acid, arachidic acid, behenic acid, lignoceric acid, cerotic acid, phosphatidic acid, phosphatidylethanolamine, phosphatidylcholine (lecithin), dodecylphosphocholine, phosphatidylserine, phosphatidylinositol, phosphatidylinositol phosphate, phosphatidylinositol bisphosphate, phosphatidylinositol triphosphate, ceramide phosphorylcholine, ceramide phosphorylethanolamine, and combinations of any of the foregoing.

39-49. (canceled)
